(19) United States
(12) Patent Application Publication
(10) Pub. No.: US 2025/0387149 A1
(43) Pub. Date: Dec. 25, 2025

(54) BONE FIXATION SYSTEM FOR EXTREMITIES AND RELATED METHODS

(71) Applicant: Forma Medical, Inc., Camp Hill, PA (US)

(72) Inventors: James A. GAULT, Lincoln, RI (US); Andrew Charles DAVISON, Hummelstown, PA (US); Christopher F. HYER, Worthington, OH (US); John Randolph CLEMENTS, Camp Hill, PA (US)

(73) Assignee: Forma Medical, Inc., Camp Hill, PA (US)

(21) Appl. No.: 19/033,786

(22) Filed: Jan. 22, 2025

Related U.S. Application Data
(60) Provisional application No. 63/623,746, filed on Jan. 22, 2024.

(51) Int. Cl. A61B 17/80; A61B 17/00; A61B 17/17; A61B 17/90; A61B 90/00

(52) U.S. Cl. CPC A61B 17/8061; A61B 17/1775; A61B 17/90; A61B 2017/00477; A61B 2090/3966

(57) ABSTRACT

A bone fixation system configured to fix one or more bone segments of extremity bones is provided.

### CROSS-REFERENCE TO RELATED APPLICATIONS

The present application claims the benefit of and priority to U.S. Provisional Application No. 63/623,746, filed Jan. 22, 2024, the entire contents of which are incorporated by reference into the present application.

### TECHNICAL FIELD

The present disclosure relates generally to a bone fixation system for extremities and related methods.

### BACKGROUND

A common procedure for handling healing of broken bones and addressing deformities such as hammertoe is the use of bone fixation implants for fusing one or more adjacent bones. Conventional bone fixation implants utilize generic screws and wires that create a rigidly fused joint with very limited adjustability intraoperatively. Some implants offer some limited degree of flexibility and/or adjustment when used under very specific circumstances that require highly technical surgical procedures. Such existing bone fixation implants often require multiple components with many intricate mating features requiring customization depending on the type of bone, patient, or desired location of the implant in the body of a patient. This results in increased costs, less desirable healing outcomes, and multiple procedures to achieve a desired outcome.

Thus, there is still a need for a bone fixation system and related surgical instruments capable of being implanted that addresses the aforementioned problems of conventional bone fixation implants including providing flexibility in bone-to-bone alignment with less invasive procedures that are pragmatic for the operating room and applicable for use with some of the smallest bones of the human anatomy.

### SUMMARY

An embodiment of the present disclosure includes a bone fixation system configured to join bone segments together. The bone fixation includes a bone implant having a proximal end, a distal end, a cannulation that extends from the proximal end to the distal end, an inner surface defining the cannulation, a proximal threaded surface at or near the proximal end, and a distal thread surface at or near the distal end and spaced from the proximal threaded surface. The proximate threaded surface is configured to engage a first bone segment and the distal threaded surface is configured to engage a second bone segment. Also includes is an internal implant core having a leading end, a trailing end opposite the leading end, and an outer surface, the internal implant core configured to be inserted into and mate with the cannulation of the bone implant.

Another embodiment of the present disclosure includes a bone fixation system configured to join bone segments together. The bone fixation system includes a proximal bone implant having a proximal end, a distal end, a proximal cannulation that extends from the proximal end to the distal end, and a proximal threaded surface configured to engage a first bone segment. The bone fixation system includes a distal bone implant having a proximal end, a distal end, a distal cannulation that extends from the proximal end to the distal end of the distal bone implant, and a distal threaded surface configured to engage a second bone segment. The bone fixation system includes a flexible elongated element having a trailing end, a leading end, and an inner channel. The trailing end is coupled to the proximal bone implant and the leading end is coupled to the distal bone implant, such that, the proximal bone implant and the distal bone implant are moveable relative to each other and the flexible elongated element.

Another embodiment of the present disclosure is joint preparation jig having a first end, a second end opposite the first end in a longitudinal direction, a first side edge, a second side edge spaced from the first side in a lateral direction that is perpendicular to the longitudinal direction, an inferior surface configured to face tissue, and a superior surface spaced from the inferior surface in a transverse direction that is perpendicular to the longitudinal direction and the lateral direction, wherein the inferior surface is curved to conform to tissue. The joint preparation includes one or more fixation slots at either or both of the first end and the second end and extending from the inferior surface to the superior surface, the one or more fixation slots further oriented in the longitudinal direction. The one or more slots are configured to receive a fixation device to temporarily secure the joint preparation jig relative to the tissue. The joint preparation jig also includes one or more guide slots extending from the inferior surface to the superior surface, the one or more guide slots oriented in a direction transverse to the longitudinal direction. The one or more guide slots are configured to receive a tissue preparation device.

Another embodiment of the present disclosure is a fixation plate having an inferior surface, a superior surface opposite the inferior surface along a transverse direction, a middle portion configured to span a joint, a first fixation portion extending from and angularly offset with respect to the middle portion, and a second fixation portion extending from and angularly offset with respect to the middle portion. The middle portion has at least one engagement member configured to engage an aiming jig. The first fixation portion has one or more through-holes that extend from the superior surface to the inferior surface. The second fixation portion having one or more through-holes that extend from the superior surface to the inferior surface. One or more anchors may be included that are configured to be inserted into the one or more through-holes to engage tissue.

Another embodiment of the present disclosure is an aiming jig having a middle portion, a first guide portion extending from and angularly offset with respect to the middle portion, and a second guide portion extending from and angularly offset with respect to the middle portion. The middle portion has an engagement member configured to facilitate temporary coupling to a fixation plate. The first guide portion has one or more channels that extend from the superior surface to the inferior surface. The second guide portion having one or more channels that extend from the superior surface to the inferior surface. The one or more channels on the first guide portion and the second guide portion configured to receive therethrough a wire. The aiming jig has an arm extension coupled to the middle portion of the aiming jig. The arm extension has a guide member having a channel that extends therethrough, the channel configured to receive a wire therethrough. A threaded post is configured to engage the engagement member and the fixation plate to couple the aiming jig to the fixation plate.

Another embodiment of the present disclosure is a bone fixation system having a preparation device. The preparation device has a preparation device body with a proximal end, a distal end spaced from the proximal end, a curved tissue facing surface to place around the tissue, an upper surface spaced from the curved tissue facing surface, and a slot extending into the body. The extends from the upper surface to the tissue facing surface. The preparation device includes an osteotomy block coupled to the device body and an osteotomy slot that extends through the osteotomy block and aligned with the slot of the preparation device body.

Another embodiment of the present disclosure is a surgical instrument having an elongated body with a first leg having a leading end, a second leg having a trailing end spaced from the leading end along and aligned with an alignment axis, and a brace that connects the first leg to the second leg. The surgical instrument has a first positioning element at the leading end, and a second positioning element that is generally aligned with the first positioning element and with the alignment axis. A cannulated actuator is configured to be movably engaged with the second positioning element. The cannulated actuator has a cannulation aligned with the alignment axis. The surgical instrument has a guide member carried by the brace. The guide member has a guide channel that extends through the guide member. The guide channel is substantially parallel to the alignment axis. The surgical instrument includes a fixation guide member carried by the brace. The fixation guide member having one or more fixation guide channels that extend in a direction that is angularly offset with respect to the alignment axis.

Another embodiment of the present disclosure is a surgical instrument having an elongated body with a first leg having a leading end, a second leg having a trailing end spaced from the leading end along and aligned with an alignment axis, and a brace that connects the first leg to the second leg. The surgical instrument has a first positioning element at the leading end, and a second positioning element that is generally aligned with the first positioning element and with the alignment axis. Also included is a driving tool movably engaged with the second positioning element, the driving tool including a head, a shaft that extends from head, and an engagement tip at a distal end of the shaft. The shaft is movably engaged with the second positioning element.

Another embodiment of the present disclosure is a bone fixation system including a support tray configured to support a plantar side of a toe. The support tray has a support body having a first end and a second end opposite the first end along an alignment axis, and an instrument engagement member coupled to the second end of the support body. The instrument engagement member has a first wall and a second wall spaced from the first wall and a first slot defined between the first and second wall. The first slot extends in a direction aligned with the alignment axis. The first slot is configured to receive an instrument therein.

Another embodiment of the present disclosure is a surgical instrument a distal guide component and a proximal guide component that is configured to be removably coupled to the distal guide component. The distal guide component has a distal component body with a distal engagement element, and a distal leg extending from the distal engagement element and defining a leading end of the distal guide component. The proximal guide component has a proximal component body with a proximal engagement element, and a proximal leg extending from the proximal engagement element and defining a trailing end of the distal guide component. The proximal engagement element is configured to couple to the distal engagement element to removably couple the distal and proximal components together. The leading end and trailing end aligned along an alignment axis when the distal and proximal components are coupled together.

Another embodiment of the present disclosure is a surgical instrument having an elongated body with a first leg having a leading end, a second leg having a trailing end spaced from the leading end along and aligned with an alignment axis, and a brace that connects the first leg to the second leg. The surgical instrument includes a first positioning element at the leading end, the first positioning element having a first guide channel, and a second positioning element. The second positioning element is generally aligned with the first positioning element and has a second guide channel align with the alignment axis and the first guide channel. The surgical instrument includes a radiopaque element carried by the elongated body. The radiopaque is configured to enable visualization of the surgical instrument.

### BRIEF DESCRIPTION OF THE DRAWINGS

The foregoing summary, as well as the following detailed description of exemplary embodiments of the present application, are better understood when read in conjunction with the appended drawings. For the purposes of illustrating the present application, there is shown in the drawings, exemplary embodiments of the disclosure. It should be understood, however, that the application is not limited to the precise arrangements and instrumentalities shown. In the drawings:

FIG. 1A is a top view of an anatomical foot;

FIG. 1B is schematic of a bone fixation system according to an embodiment of the present disclosure;

FIG. 2 illustrates a method of using the bone fixation system in FIG. 1B;

FIG. 3 is a bone implant according to an embodiment of the present disclosure;

FIG. 4 is a driver used for the implant in shown in FIG. 3;

FIG. 5 illustrates the driver shown in FIG. 3 engaged with the bone implant shown in FIG. 2;

FIG. 6-8 illustrate a joint preparation jig of a bone fixation system according to another embodiment of the present disclosure;

FIGS. 9-13B illustrate a bone fixation plate used of a bone fixation system according to another embodiment of the present disclosure;

FIGS. 14-16 illustrate an aiming jig used with the bone fixation plate shown in FIGS. 9-13B;

FIG. 17 illustrates the aiming jig shown in FIGS. 14-16 coupled to the bone fixation plate shown in FIGS. 9-13B;

FIGS. 18-21 illustrates a preparation device of a bone fixation system according to another embodiment of the present disclosure;

FIGS. 22 and 23 illustrates a method of using the preparation device shown in FIGS. 18-21;

FIGS. 24-26 illustrates a preparation device according to another embodiment of the present disclosure;

FIGS. 27-30 illustrate a surgical instrument according to another embodiment of the present disclosure;

FIGS. 31-32 illustrate a surgical instrument according to another embodiment of the present disclosure;

FIGS. 33-35 and 38 illustrates a surgical instrument according to another embodiment of the present disclosure;

FIGS. 36 and 37 illustrates an actuator used with the surgical instrument shown in FIGS. 33-35;

FIG. 39 illustrates the surgical instrument shown in FIGS. 33-35 used with a support tray according to an embodiment of the present disclosure;

FIGS. 40-41 illustrate a surgical instrument according to another embodiment of the present disclosure;

FIGS. 42-43 illustrate a surgical instrument according to another embodiment of the present disclosure;

FIGS. 44-48 illustrate a surgical instrument according to another embodiment of the present disclosure;

FIG. 49 is a side view of a bone fixation system in accordance with an exemplary embodiment of the present disclosure;

FIG. 50 is an exploded side view of the bone fixation system shown in FIG. 49;

FIG. 51 is a perspective view of a bone anchor used with the bone fixation system shown in FIGS. 49 and-51;

FIG. 52 is a side view of an implant and wire of a bone fixation system being inserted into a phalange in accordance with an exemplary embodiment of the present disclosure;

FIG. 53 is a side view of the implant and wire inserted into the phalange shown in FIG. 15;

FIG. 54 is a side view of the implant inserted into the phalange shown in FIG. 15 with the wire removed; and

FIG. 55 is a side view of a driver of a bone fixation system in accordance with an exemplary embodiment of the present disclosure.

### DETAILED DESCRIPTION OF ILLUSTRATIVE EMBODIMENTS

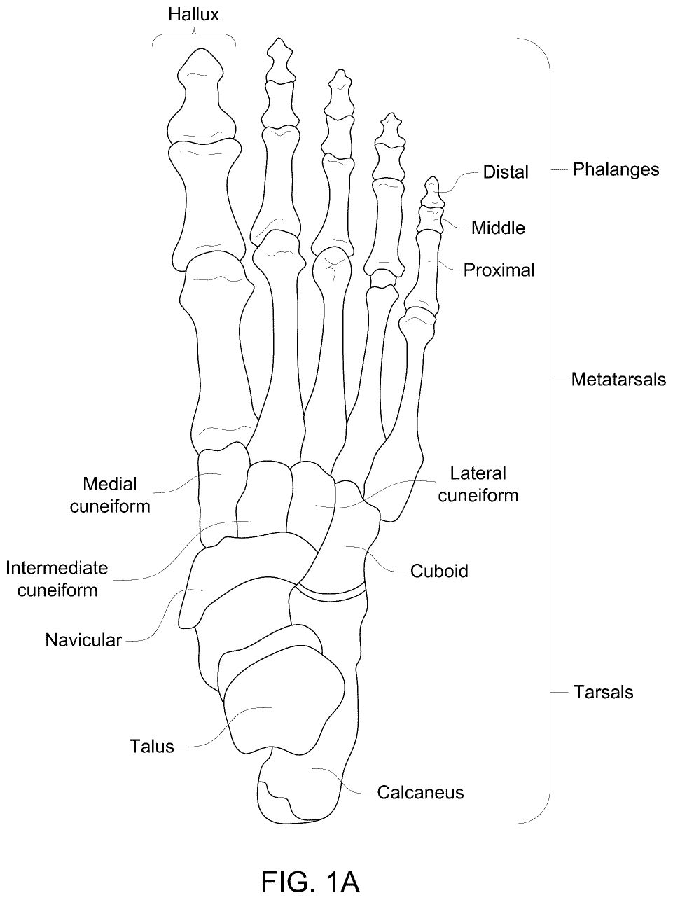

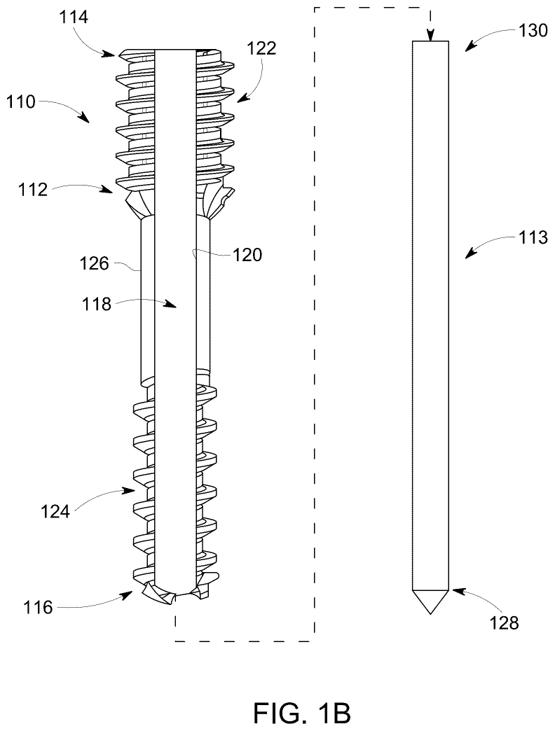

Bone fixation devices as described are configured for aid in the fixation of two or more bones or bone segments, typically in extremity bones, such as the foot. As shown in FIG. 1A, the skeletal anatomy of a foot includes tarsals, metatarsals, and phalanges. The foot bone structure is further typically divided into three regions: the hindfoot, midfoot, and forefoot. The tarsal bones are seven bones in the hindfoot and midfoot and include the calcaneus, talus, cuboid, navicular, and three cuneiforms. The metatarsal bones are five bones in the forefoot that connect the tarsals to the phalanges. The phalanges in the forefoot that the toes.

The bone fixation systems and devices as described herein are configured for fixation of bone structure in the foot. In other examples, the bone fixation system and devices are configured for interphalangeal joint fixation. For example, the bone fixation devices may be used for fixation of metatarsals, proximal phalanges, middle phalanges, or distal phalanges. While the embodiments described are configured for interphalangeal joint fixation, it is possible that the described embodiments could be configured for fixation of phalanges, metatarsals, cuneiform, or cuboid bones in the foot. In other embodiments, the bone fixation devices may be configured for fixation of bone segments of phalanges, metatarsals or other bones in the hand.

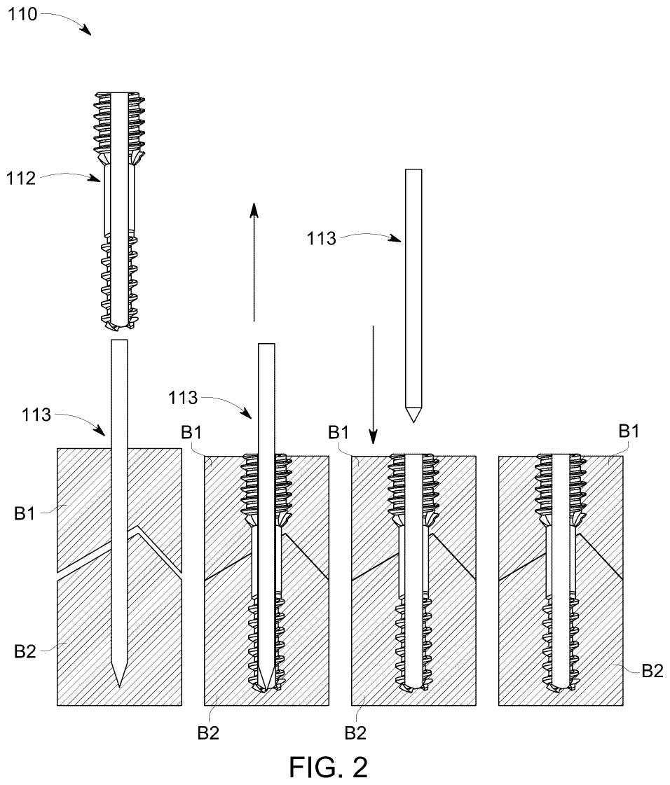

Referring to FIGS. 1B-2, there is shown an exemplary embodiment of a bone fixation system 110 configured to join bone segments together. The bone fixation system includes a bone implant 112 and an internal implant core 113. The bone implant 112 has a proximal end 114, a distal end 116, a cannulation 118 that extends from the proximal end 114 to the distal end 116, an inner surface 120 defining the cannulation 118, a proximal threaded surface 122 at or near the proximal end 114, and a distal thread surface 124 at or near the distal end 116 and spaced from the proximal threaded surface 122. The proximal threaded surface 122 is configured to engage a first bone segment and the distal threaded surface 124 is configured to engage a second bone segment. In yet another example, the bone implant 112 further comprises an unthreaded outer surface 126 that extends from the proximal threaded surface 122 to the distal threaded surface 124. Alternatively, an entirety of the outer surface of the bone implant 112 is threaded. The bone implant 112 may be a metal, metal alloy or alloys or a polymeric material (e.g. such PGA or other similar polymers). In another example, the internal bone implant 113 is bioabsorbable.

The internal implant 113 core has a leading end 128, a trailing end 130 opposite the leading end 128, and an outer surface 132. Here, the internal implant core 113 is configured to be inserted into and mate with the cannulation 118 of the bone implant 112. The internal implant core 113 may be a metal or metal alloy or a polymeric material (e.g. such PGA or other similar polymers). In another example, the internal implant core 113 is bioabsorbable.

The bone implant 112 and internal implant core 113 are configured to mate and engage with each other during use. For example, the inner surface 120 of the bone implant 112 has a first cross-sectional dimension C1, and the outer surface 132 of the internal implant core 113 has a second cross-sectional dimension C2. The first-cross sectional dimension C1 is greater than or equal to the second cross-sectional dimension C2. In addition, the bone implant 112 has a first length that extends from the proximal end 114 to the distal end 116, and the internal bone implant 113 has a second length that extends from the leading end 128 to the trailing end 130. In this case, the second length is the same or greater than the first length.

In one example, the inner surface 120 of the bone implant 112 is threaded, and the outer surface of the internal implant core 113 is threaded, such that, the internal bone implant 113 is configured to be threaded into the cannulation 118 of the bone implant 112. In another example, the inner surface 120 of the bone implant 112 is not threaded, and the outer surface of the internal implant core 113 is not threaded, such that, the internal implant core 113 is configured to be slide into the cannulation 118 of the bone implant 112.

As shown in FIG. 2, the bone fixation system 110 may be used to joint to bone segments B1, B2 together. In use, such a surgical method includes inserting a wire (not shown) into a target site until its distal end (not shown) contacts a first bone segment. The method also includes implanting a threaded bone implant 112, that has a cannulation 118, over the wire until a distal threaded surface 124 of the bone implant 112 threadably engages the first bone segment, and a proximal threaded surface 122 of the bone implant 112 threadably engages a second bone segment. Next, the use may remove the wire from the cannulation 118 of the bone implant 112. Then, the user may insert a leading end 128 of an internal bone implant 113 into a proximal opening (not numbered) of the cannulation 118 of the bone implant 112.

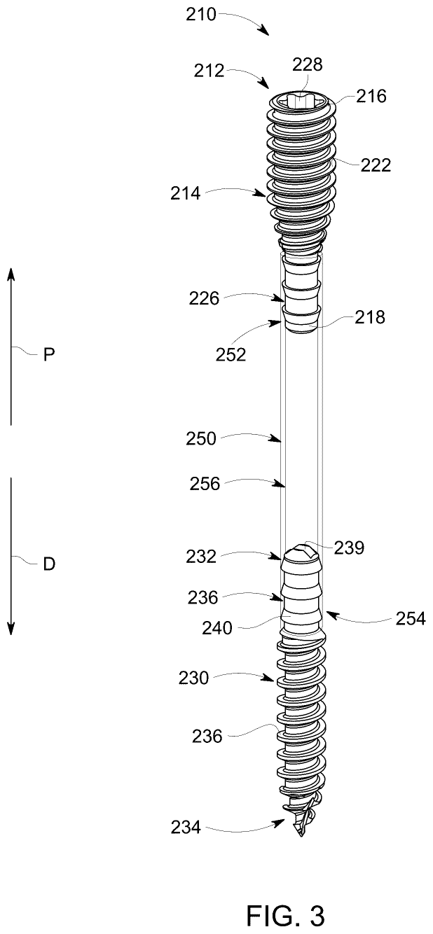

FIGS. 3-5 illustrate a bone fixation system 210 configured to join bone segments according to another embodiment of the present disclosure. The bone fixation system 210 includes an implant system 212, which includes a flexible elongated element 250, and a driver 280. More specifically, the bone fixation system 210 includes a proximal bone implant 214 with a proximal end 216, a distal end 218, a proximal cannulation 220 that extends from the proximal end 216 to the distal end 218, and a proximal threaded surface 222 configured to engage a first bone segment. In one example, the proximal bone implant 212 has an inner surface 224 defining the proximal cannulation 220 and a distal engagement surface 226 at or near the distal end 218 and spaced from the proximal threaded surface 222 in a distal direction D. The proximal end 216 of the proximal bone implant 214 defines an engagement feature 228 configured to engage the driver 280.

The bone fixation system 210 also includes a distal bone implant 230 has a proximal end 232, a distal end 234, a distal cannulation 235 that extends from the proximal end 232 to the distal end 234 of the distal bone implant 230, and a distal threaded surface 236 configured to engage a second bone segment. The distal bone implant 230 has an inner surface 238 defining the distal cannulation 235 and a proximal engagement surface 240 spaced from the distal threaded surface 236 in a proximal direction P that is opposite the distal direction D. In one example, the pitch of threads of the proximal threaded surface 222 of the proximal bone implant 214 is different from a pitch of threads of the distal threaded surface 236 (described below). In another example, the of threads of the proximal threaded surface 222 is substantially the same as a pitch of threads of the distal threaded surface 236. Furthermore, the proximal end 232 of the distal bone implant 230 defines an engagement feature 239 configured to engage the driver 280. For example, the driver 280 can include single shaft that has separate features that engage the proximal bone implant 222 and the distal bone implant 230.

In addition, the bone fixation system 210 includes a flexible elongated element 250 has a trailing end 252, a leading end 254, and an inner channel 256. The trailing end 252 is coupled to the proximal bone implant 214 and the leading end 254 is coupled to the distal bone implant 230, such that, the proximal bone implant 214 and the distal bone implant 230 are moveable relative to each other and the flexible elongated element 250. More specifically, the trailing end 252 engages the distal engagement surface 226 of proximal bone implant 214, and the leading end 254 engages the proximal engagement surface 240 of the distal bone implant 230. The flexible elongated element 250 may be a polymeric tube. Alternatively, the flexible elongated element 250 could be a braided wire. Thus, the flexible elongated element 250 could be any elongated element that is flexible includes an inner channel or cannulation that permits a driver or other devices, such a wire, etc. to pass through the flexible elongated element 250.

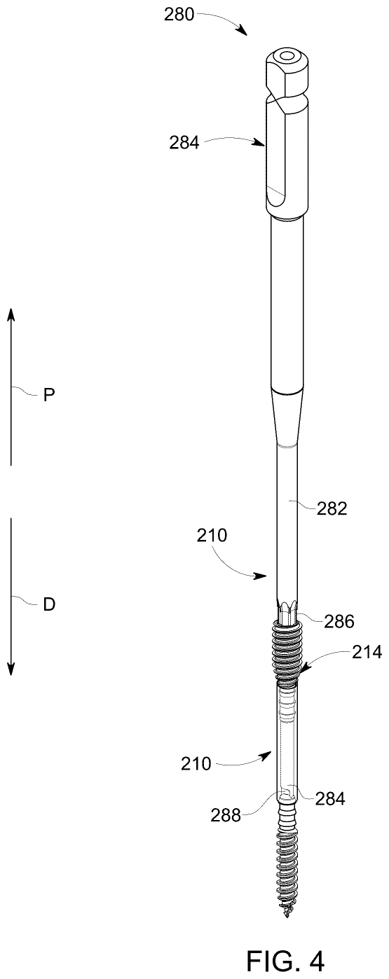

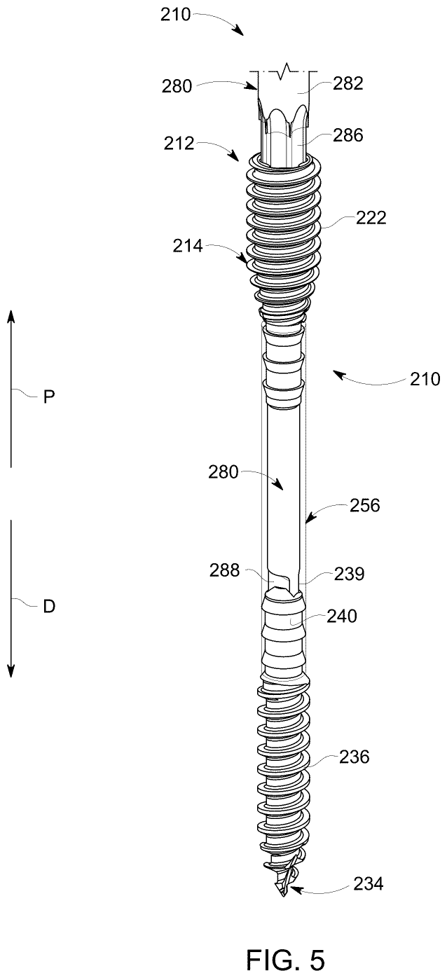

As shown in FIGS. 4 and 5, the driver 280 has a shaft 282 with a proximal end 284, a proximal engagement element 286, and a distal engagement element 288 opposite the proximal end 284 spaced apart from the proximal engagement element 286. The driver 280 is insertable through the proximal cannulation of the proximal bone implant 214 and the inner channel of the flexible elongated element 250 so that the proximal engagement element 286 engages the proximal end 216 of the proximal bone implant 214, and the distal engagement element 288 engages the proximal end 232 of the distal bone implant 230.

In use, bone fixation system 210 may be used to join to bone segments together. The method includes inserting a bone implant system 210 into a target site until a distal bone implant 230 threadably engages a first bone segment and a proximal bone implant 214 threadably engages a second bone segment. Next, the user can drive the distal bone implant 230 into a bone segment with a distal engagement element 288 of the driver 280 and can drive the proximal bone implant 214 into another bone segment with a proximal engagement feature 286 of the driver 280.

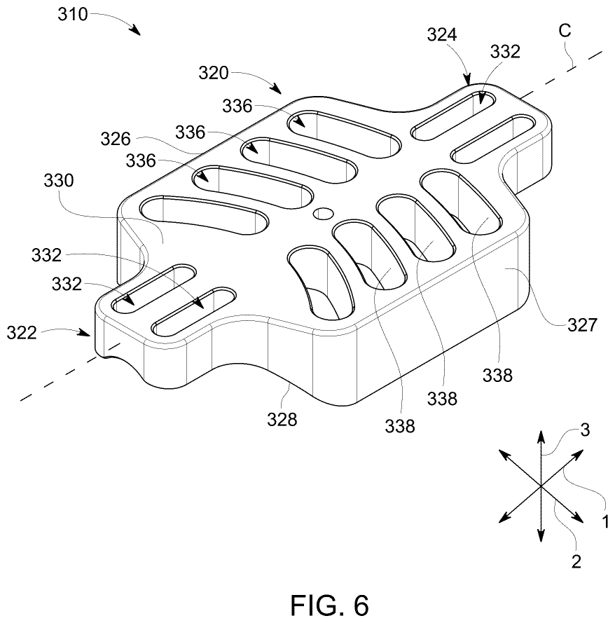

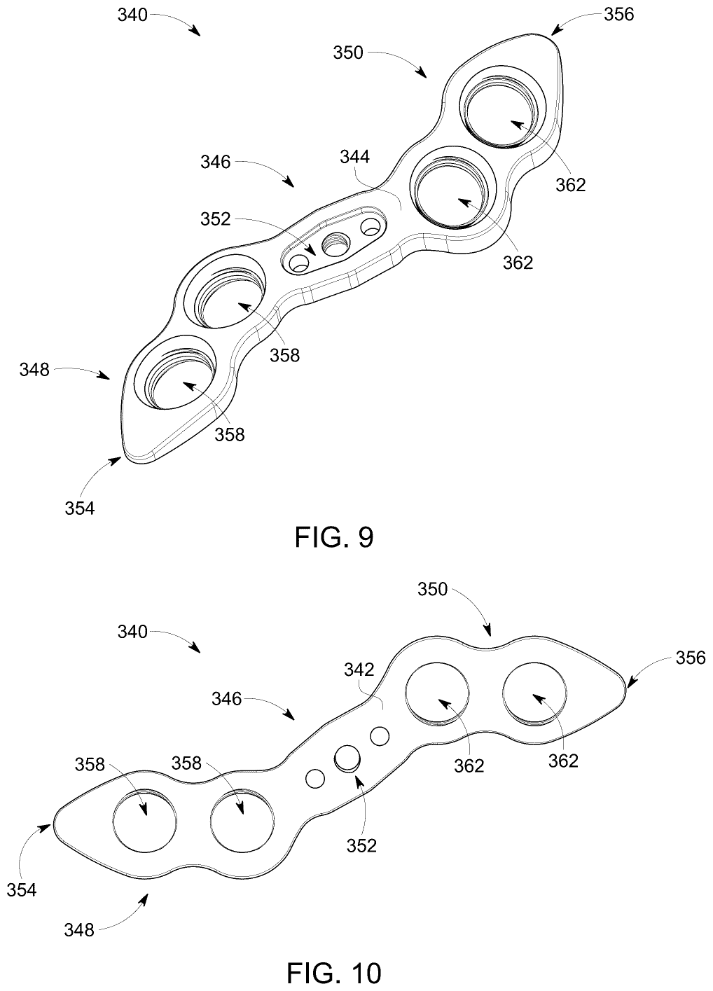

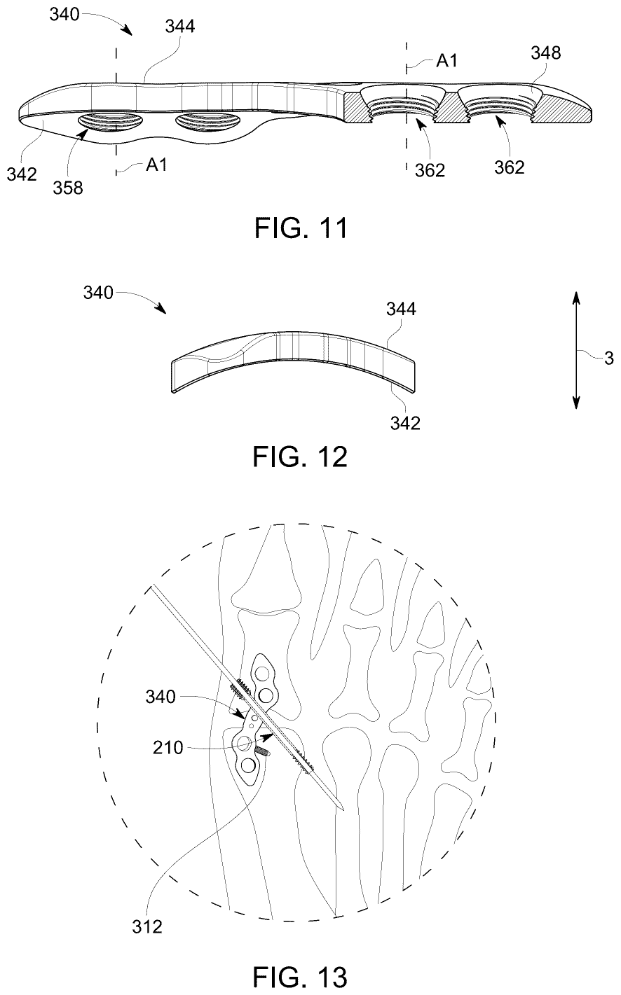

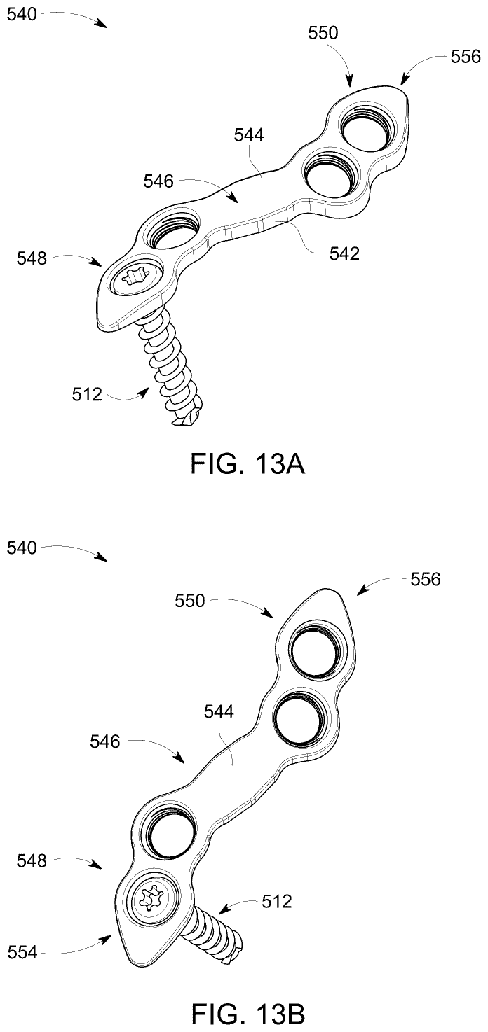

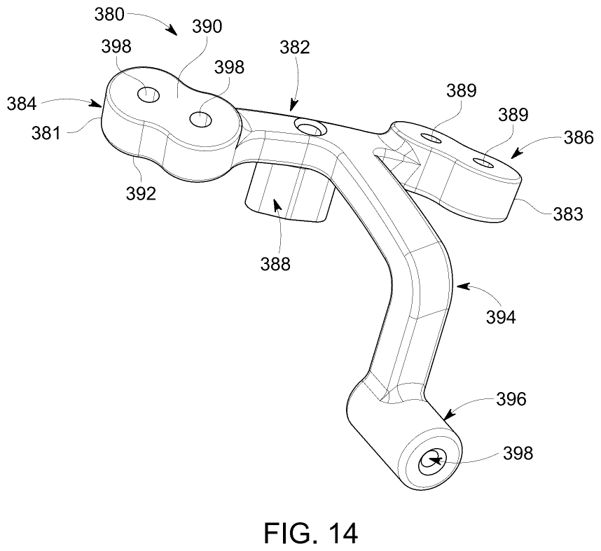

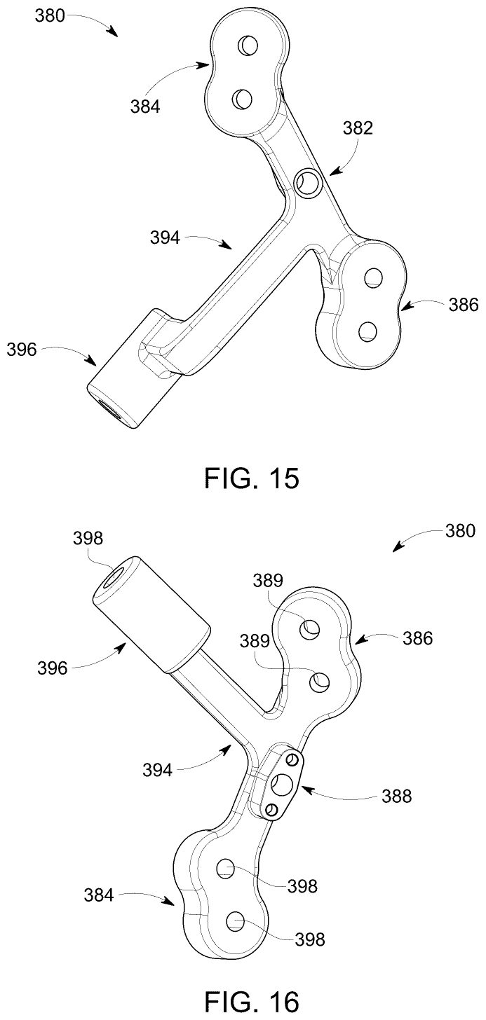

FIGS. 6-17 illustrate a bone fixation system 310 according to another embodiment of the present disclosure. The bone fixation system includes a joint preparation jig 320 (FIGS. 6-8), a fixation plate 340 (FIGS. 9-12), and an aiming jig 380 (FIGS. 14-17). The bone fixation system 310 also includes one or more bone anchors 312 (FIG. 13, 13A, and 13B) configured to be inserted into the one or more through-holes of the fixation plate 340 to engage tissue.

Turning to FIGS. 6-8, the joint preparation jig 320 has a first end 322, a second end 324 opposite the first end 322 in a longitudinal direction 1, a first side edge 326, a second side edge 327 spaced from the first side edge 326 in a lateral direction 2 that is perpendicular to the longitudinal direction 1, an inferior surface 328 configured to face tissue, and a superior surface 330 spaced from the inferior surface 328 in a transverse direction 3 that is perpendicular to the longitudinal direction 1 and the lateral direction 2. The inferior surface 328 is curved to conform to tissue, such as skin or bone.

The jig 320 has one or more fixation slots 332 at either or both of the first end 322 and the second end 324 and extending from the inferior surface 328 to the superior surface 330. As shown, the fixation slots 332 further oriented in the longitudinal direction 1. The slots 332 are configured to receive a fixation device (not numbered or shown in FIGS. 6-8) to temporarily secure the joint preparation jig 320 relative to the tissue. The fixation slots 332 include a first pair of fixation slots 332 at the first end 322, and a second pair of fixation slots 332 at the second end 324.

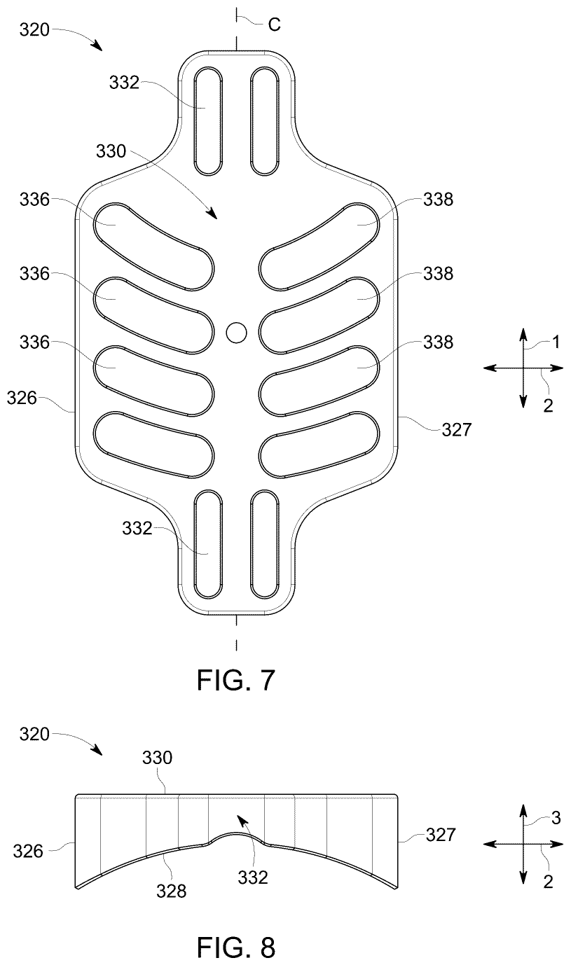

The jig 320 has one or more guide slots 334 extending from the inferior surface 328 to the superior surface 330. The guide slots 334 are oriented in a direction that is transverse to the longitudinal direction 1. The guide slots 334 are configured to receive a tissue preparation device (not shown in FIGS. 6-8). In one example the guide slots 334 include a first set 336 of four guide slots between the first side edge 326 and a central axis C that extends in the longitudinal direction 1, and a second set 338 of four guide slots between the second side edge 327 and the central axis C and opposite the first set 336 of four guide slots. Thus, as shown, there are eight guide slots but there could two guide slots, three guide slots, four guide slots, including any number up to eight guide slots. In some cases, depending on the size of the jig 320, there could be more than eight guide slots 334. In addition, one or more of the first set 336 of four guide slots are curved, and one or more of the second set 338 of four guide slots are curved, as shown in FIG. 7.

Referring to FIGS. 9-13, the bone implant is configured as a fixation plate 340 for securing bone segments together. The fixation plate 340 has an inferior surface 342, a superior surface 344 opposite the inferior surface 342 along a transverse direction 3, a middle portion 346 configured to span a joint, a first fixation portion 348 extending from and angularly offset with respect to the middle portion 346, and a second fixation portion 350 extending from and angularly offset with respect to the middle portion 346. In the example shown, the middle portion 346 has at least one engagement member 352 configured to engage an aiming jig 380 (described below). The implant 340 may have a first end 354 at the first fixation portion 348 that is tapered, and a second end 356 at the second fixation portion 350 that is tapered. The first and second tapered ends 354, 356 are configured to guide the fixation plate 340 between skin and bone proximate a joint.

The first fixation portion 348 has one or more through-holes 358 that extend from the superior surface 344 to the inferior surface 342. The through-holes 358 of the first fixation portion 348 extends along a through-hole axis A1 and include an upper portion 360 that is tapered inward toward the through-hole axis A. In the example shown, the first fixation portion 348 includes two through-holes, though more or less through-holes may be used. The second fixation portion 350 has one or more through-holes 362 that are configured like the through-holes 358 of the first fixation portion 348. Accordingly, the through-holes 362 that extend from the superior surface 344 to the inferior surface 342. The through-holes 362 of the second fixation portion 350 extends along a through-hole axis and include an upper portion that is tapered inward toward the through-hole axis. In the example shown, the second fixation portion 350 includes two through-holes, though more or less through-holes may be used.

FIGS. 13A and 13B illustrate another embodiment of the fixation plate 540. As shown, the fixation plate 540 as shown in in FIGS. 13A and 13B is configured like the fixation plate 540 as shown in FIGS. 9-13. As shown, the fixation plate 540 has an inferior surface 542, a superior surface 544 opposite the inferior surface 542 along a transverse direction 3, a middle portion 546 configured to span a joint, a first fixation portion 548 extending from and angularly offset with respect to the middle portion 540, and a second fixation portion 550 extending from and angularly offset with respect to the middle portion 546 with a length that is generally greater than a length of each fixation portion 548 and 550. In the example shown, the implant 540 may have a first end 554 at the first fixation portion 548 that is tapered, and a second end 556 at the second fixation portion 550 that is tapered. The first and second tapered ends 554, 556 are configured to guide the fixation plate 540 between skin and bone proximate a joint. Each fixation portion has one or more through holes as needed to receive bone anchors 512, such as bone screws. The bone anchors 512 may be typical anchors used in fixation and include head and shaft, all or a portion of which is threaded. The bone anchors can be a locking screw or a compression screw and the through-holes may be configured to receive and engage such differing screws as needed.

Referring to FIGS. 14-16, a system 310 includes an aiming jig 380 is configured to guide a drill or other instrument toward the bone segments that the plate 340 is intended to be fixed to. The aiming jig 380 has a middle portion 382, a first guide portion 384 extending from and angularly offset with respect to the middle portion 382, a second guide portion 386 extending from and angularly offset with respect to the middle portion 382, a superior surface 390 and an inferior surface 392 opposite the superior surface 390. The jig 380 includes a first end 381 and second end 383. The middle portion 382 includes an engagement member 388 configured to facilitate temporary coupling to a fixation plate 340. The first guide portion 384 has one or more channels 398 that extend from the superior surface 390 to the inferior surface 392. In one example, the first guide portion 384 has two channels. However, one or more than two channels may be present. The second guide portion 386 has one or more channels 389 that extend from the superior surface 390 to the inferior surface 392. In one example, the second guide portion 386 has two channels. However, one or more than two channels may be present. The channels on the first guide portion 384 and the second guide portion 386 configured to receive therethrough a wire (not shown).

The aiming jig 380 includes an arm extension 394 coupled to the middle portion 382 of the aiming jig 380. The arm extension 394 has a guide member 396 that has a channel 398 that extends therethrough. The channel 398 is configured to receive a wire therethrough. A threaded post 399 is configured to engage the engagement member 388 and the bone implant 340 to couple the aiming jig 380 to the bone implant 340.

In use, a method of fixing a bone can include use of jig 320, plate 340 and aiming jjg 380. The user can place a joint preparation jig 320 along a joint in an extremity bone such that its inferior surface 328 faces surgical site of the joint. The user can insert one or more fixation wires (not shown) through one or more fixation slots 332, respectively, located a first end 322 or second end 324 of the joint preparation jig 380, until the fixation wire couples the joint preparation jig 320 to the surgical site. The user can insert a drill through a guide slot on the joint preparation jig 320 to prepare the joint for fixation. Next, the method includes removing the drill from the guide slot 334 and then removing the one or more fixation wires from the surgical site and the one or more fixation slots 332.

The method also includes forming an incision at a joint of an extremity bone. The method also includes inserting a first tapered end 354 of a fixation plate 340 into the incision in a first direction between skin and bone until a first fixation portion 348 is located adjacent a first bone of the joint. The user can then pull the fixation plate 340 in a second direction that is opposite the first direction until a middle portion 346 of the fixation plate 340 spans the joint and a second fixation portion 350 of the plate 340 is located adjacent to a second bone of the joint.

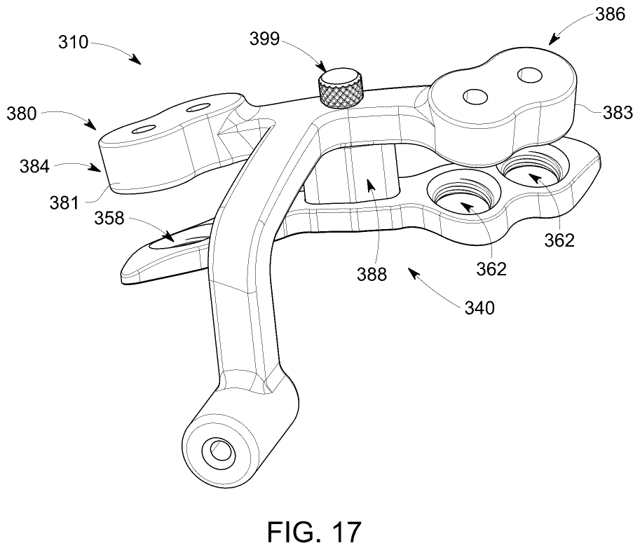

As shown in FIG. 17, the method further includes coupling the aiming jig 380 to the middle portion 382 of the fixation plate 340. The user can insert a first k-wire (not shown) through a first channel 398 of the aiming jig and through a through-hole 358 on the first fixation portion 348 of the fixation plate 340. Through-hole 358 may be referred to as a first through-hole. The method then includes implanting a first bone anchor over the first k-wire (anchor and K-wire not shown until the bone anchor engages the first bone and the through-hole 358 of the fixation plate 340. The method also includes inserting a second k-wire through a second channel 389 of the aiming jig 380 and through a through-hole 362 on the second fixation portion 350 of the fixation plate 340. Through-hole 362 may be referred to a second through-hole. The method includes implanting a second bone anchor over the second k-wire (bone anchor and k-wire not shown) until the second bone anchor engages the second bone and the second through-hole 363 of the fixation plate 340.

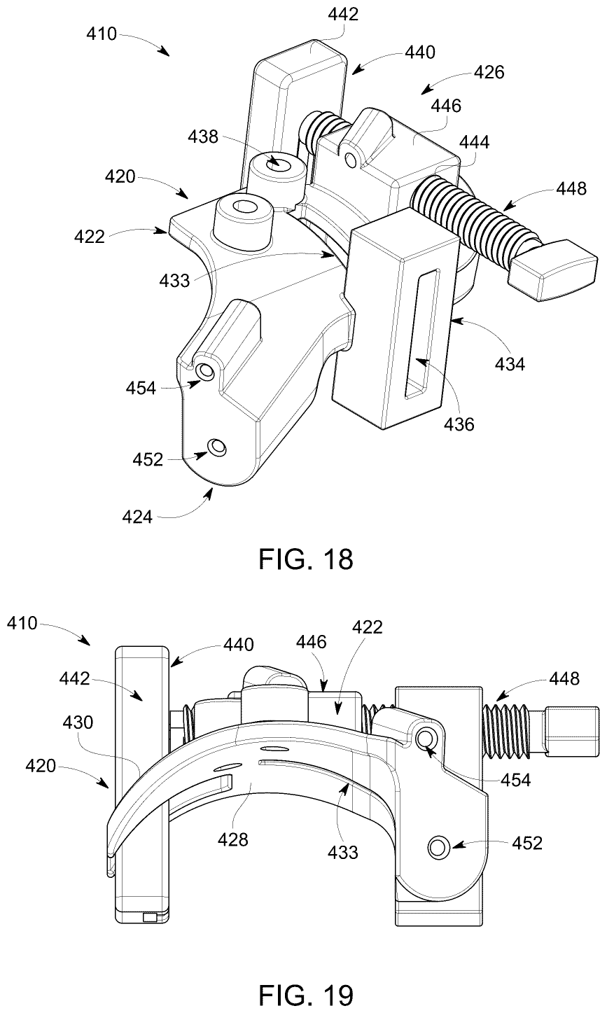

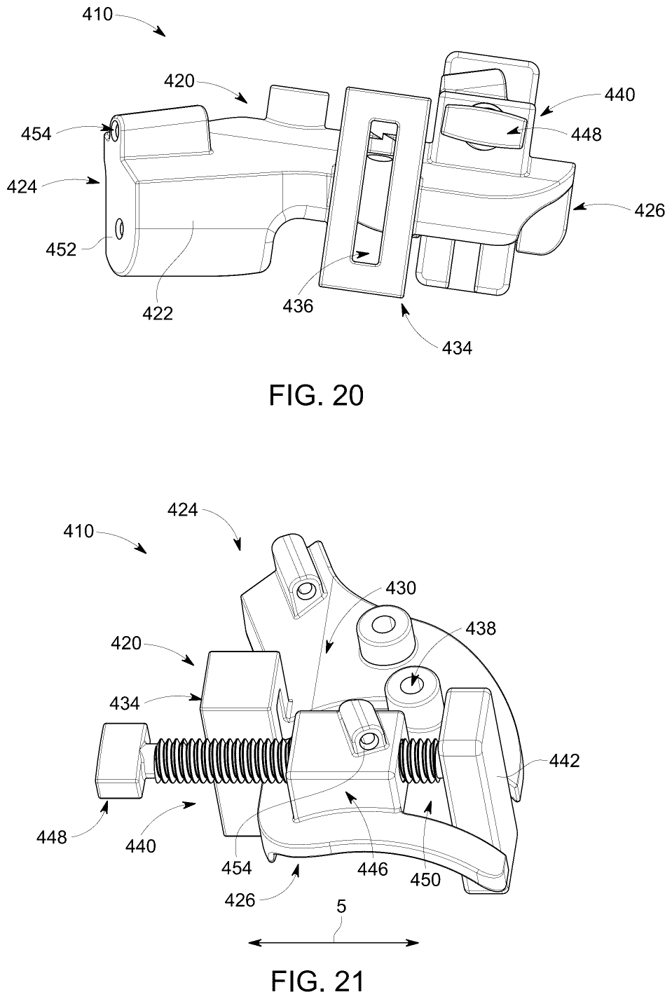

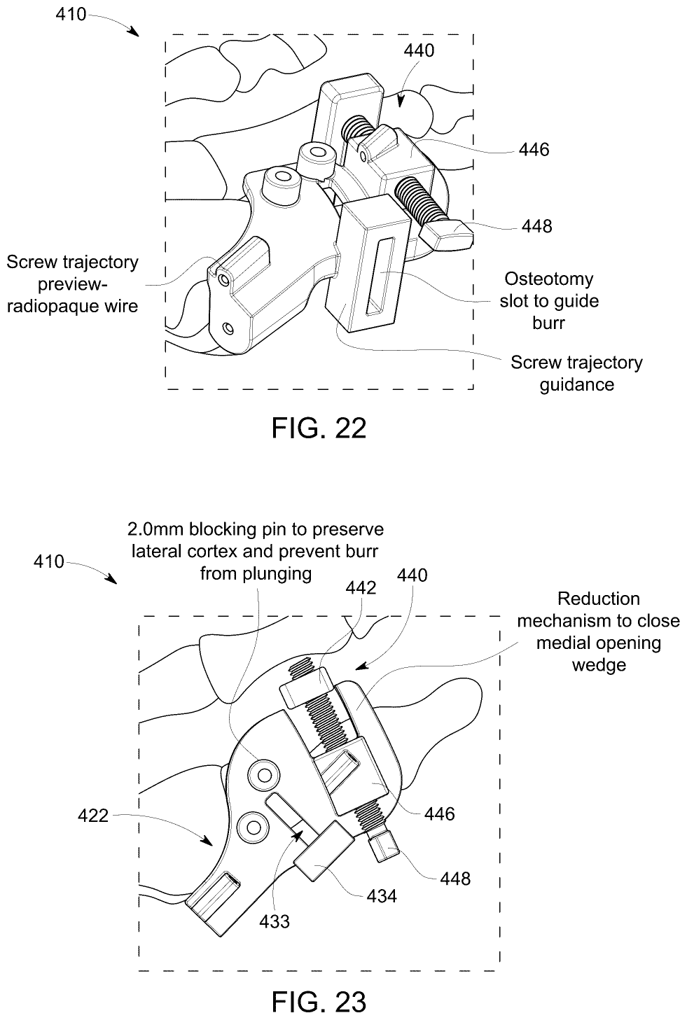

Referring to FIGS. 18-23, a bone fixation system 410 includes a preparation device 420. As shown in FIGS. 18-21, the preparation device 420 has a preparation device body 422 with a proximal end 424, a distal end 426 spaced from the proximal end 424, a curved tissue facing surface 428 to place around the tissue, an upper surface 430 spaced from the curved tissue facing surface 432, and a slot 436 extending into the preparation device body 422. In this example, the slot 433 extends from the upper surface 430 to the tissue facing surface 432. The preparation device 420 includes an osteotomy block 434 coupled to the preparation device body 422 and an osteotomy slot 436 that extends through the osteotomy block 434 and aligned with the slot 433 of the preparation device body 422. The preparation device 420 includes a block guide channel 438 aligned with the osteotomy slot 436 but also extending from surface 428 to surface 430 as shown. The block guide channel 438 configured to receive therein a wire (not shown). There may be one channel 438 or two or more channels 438 as shown. Also included is a reduction member 440 coupled to the preparation device body 422. The reduction member 440 includes a reduction block 442, a support block 446 extending from the upper surface 430 of the preparation device body 422, a threaded hole 444 in the support block 446, and an actuator 448 threadably coupled to the support block 446 and the reduction block 442. In one example, the actuator 448 may be a threaded actuator. Here, actuation of the actuator 448 causes the reduction block 442 to translate in a reduction slot 450 (FIG. 21) in the preparation device body 422 in a direction toward 5 or away from the support block 446.

The preparation device 420 also includes a fixation guide channel 452 along the curved tissue facing surface 432. The fixation guide channel 452 is configured to receive therethrough a fixation device (not shown). A site line guide channel 454 extends along an upper surface 430 of the preparation device body 422. The site line guide channel 454 may be substantially aligned with the fixation guide channel 452 and configured to receive therethrough a radiopaque wire (not shown).

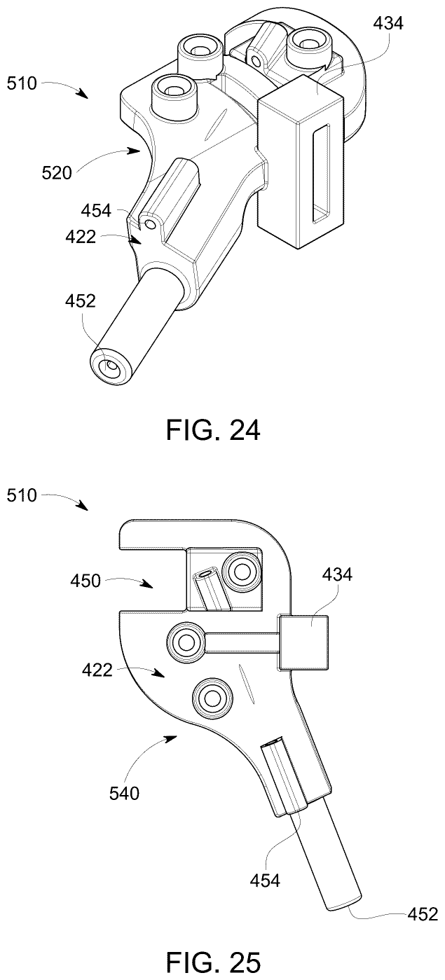

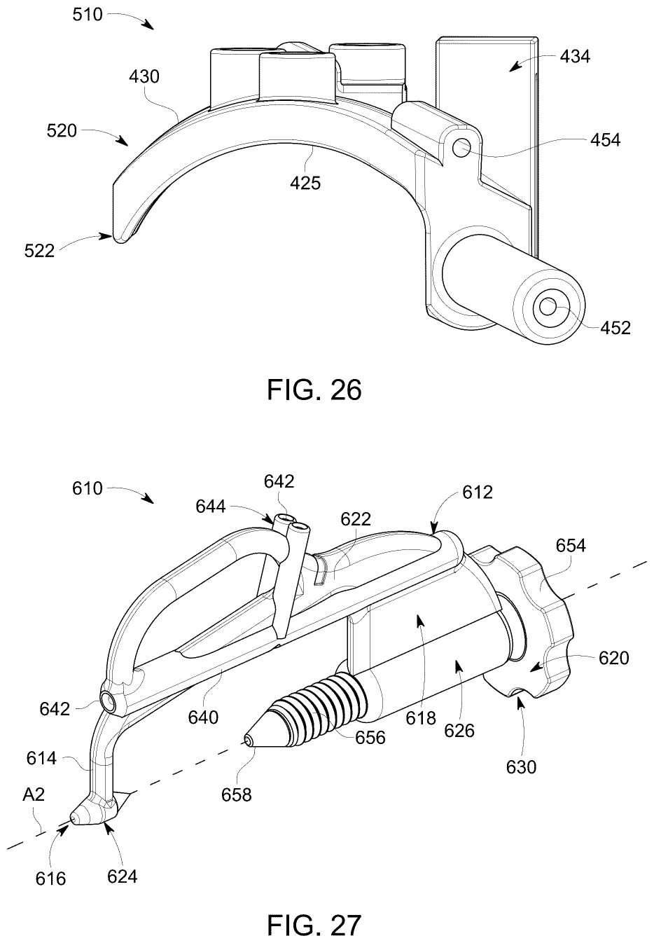

Referring to FIGS. 24-26, another embedment of a bone fixation system 510 is shown with an alternative preparation device 520. The preparation device 520 is substantially like the preparation device 420 except that the preparation device 520 does not include a reduction member. The preparation device 520 has substantially the same features as described above with respect to preparation device 420 and will not be repeated here for clarity and conciseness. The bone fixation system 510 may be used for a so-called Akin procedure used in toe fixation.

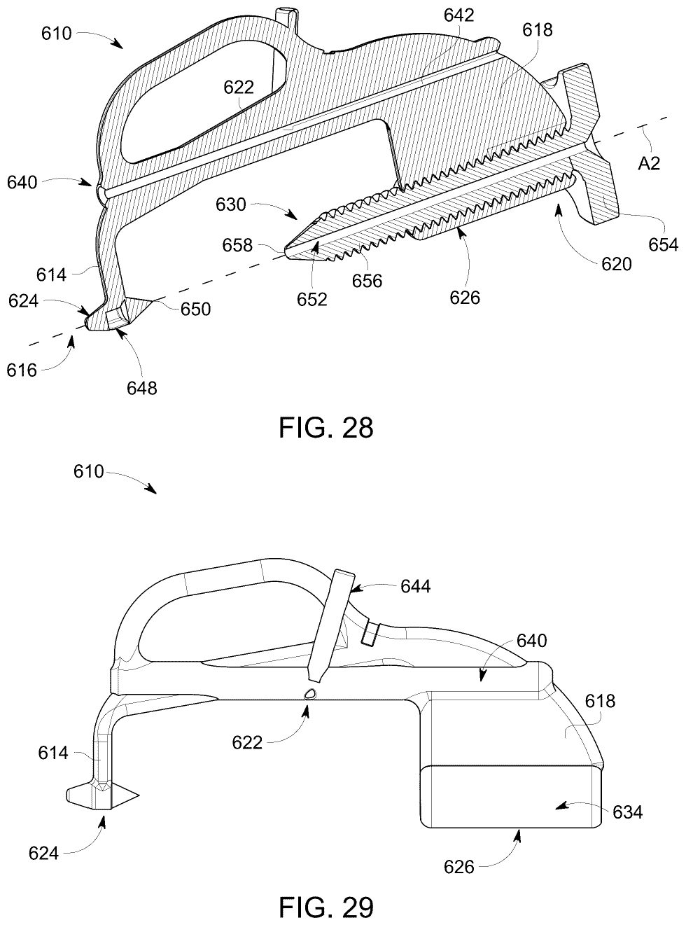

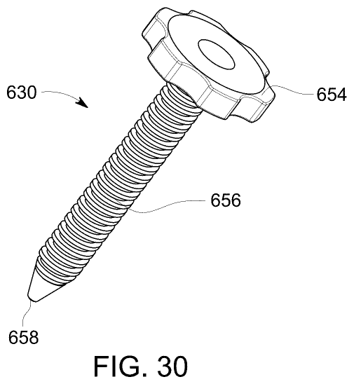

FIGS. 27-30 illustrate a surgical instrument 610 according to an embodiment of the present disclosure. The surgical instrument 610 has an elongated body 612 with a first leg 614 that has a leading end 616, a second leg 618 that has a trailing end 620 that is spaced from the leading end 616 along and aligned with an alignment axis A2, and a brace 622 that connects the first leg 614 to the second leg 618. The surgical instrument 610 also includes a first positioning element 624 at the leading end 616, and a second positioning element 626 that is generally aligned with the first positioning element 624 and with the alignment axis A2. The first positioning element 624 has an engagement member 648 including an apex 650 oriented toward the second positioning element 626. The apex 650 is substantially aligned with the alignment axis A2. The second positioning element 632 includes a support body 634 that has a threaded inner surface 636. The instrument 610 has a cannulated actuator 630 that includes a threaded outer surface 638 configured to threadably engage the threaded inner surface 636 of the support body 634.

The surgical instrument 610 also includes a guide member 640 carried by the brace 622. The guide member 640 has a guide channel 642 that extends through the guide member 640. The guide channel 642 is substantially parallel to the alignment axis A2. There may be two or more channels 642. In addition, a fixation guide member 644 is carried by the brace 622. The fixation guide member 644 has one or more fixation guide channels 646 that extend in a direction 6 that is angularly offset with respect to the alignment axis A2.

As shown in FIGS. 27, 28 and 30, the cannulated actuator 630 is movably engaged with the second positioning element 626. Here, the cannulated actuator 630 has a cannulation 652 aligned with the alignment axis A2. The cannulated actuator 630 incudes a gripping head 654, a shaft 656 that extends from the gripping head 654 toward a distal apex 658. The cannulation 652 is configured to receive therein a wire (not shown). The surgical instrument 610 according to an embodiment of the present disclosure as described herein can be used for bone fixation procedures, such as the Akin procedure.

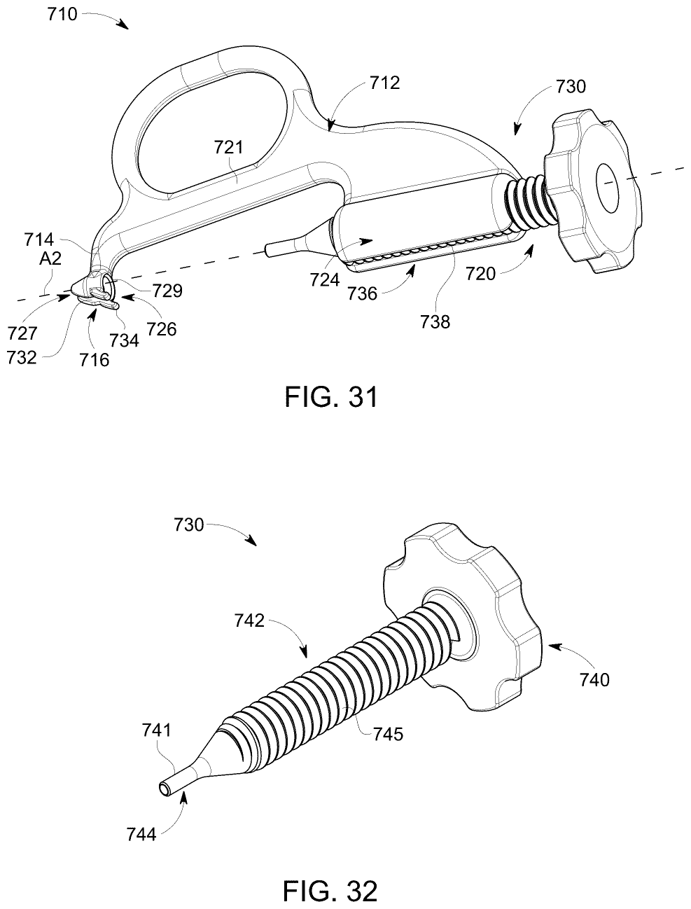

Referring to FIGS. 31 and 32, the bone fixation system includes a surgical instrument 710 and a driving tool 730 according to another embodiment of the present disclosure. The surgical instrument 710 has an elongated body 712 with a first leg 714 that has a leading end 716, a second leg 718 has a trailing end 720 spaced from the leading end 716 along and aligned with an alignment axis A2, and a brace 721 that connects the first leg 714 to the second leg 718. The surgical instrument 710 includes a first positioning element 722 at the leading end 716, and a second positioning element 724 that is generally aligned with the first positioning element 722 and with the alignment axis A2. The first positioning element 722 has an engagement member 726 including a conical shaped inner surface 729 sized and shaped to receive a head of a bone anchor, and slot 732 that extends into an inner space of the first positioning element 722 and one or more projections 734 to receive the head of the anchor. The second positioning element 724 includes a slot 736 that extends along its length. In addition, the second positioning element 724 includes a threaded inner surface 738.

The driving tool 730 may be movably engaged with the second positioning element, as shown in FIG. 31. The driving tool 730 has a head 740, a shaft 742 that extends from the head 740, and an engagement tip 741 at a distal end 744 of the shaft 742. In one example, the shaft 742 of the driving tool 730 as a threaded outer surface 745 that threadably engages with the threaded inner surface 738 of the second positioning element 724 In one example, the engagement tip 741 of the driving tool 730 is a drill bit. As such the engagement tip 741 is configured to engage a head of a bone anchor or screw to drive the anchor along the alignment axis A2.

Another embodiment of a driving tool (not shown), which is substantially similar to the driving tool 730, not shown, includes a proximal end, a shaft extending from the proximal end, a distal end opposite the proximal end. This alternative driving tool, however, includes a cannulation extending through the driving tool, a first shoulder portion on the shaft configured to engage a stop member in the second positioning element 724 to inhibit further advancement of the driving tool through the second positioning element 724.

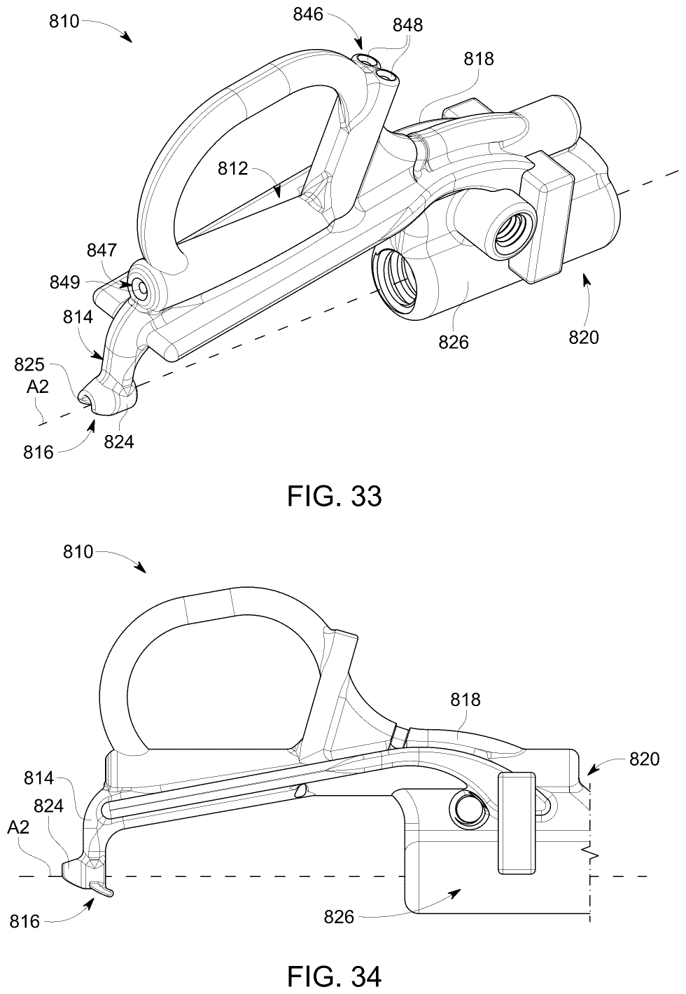

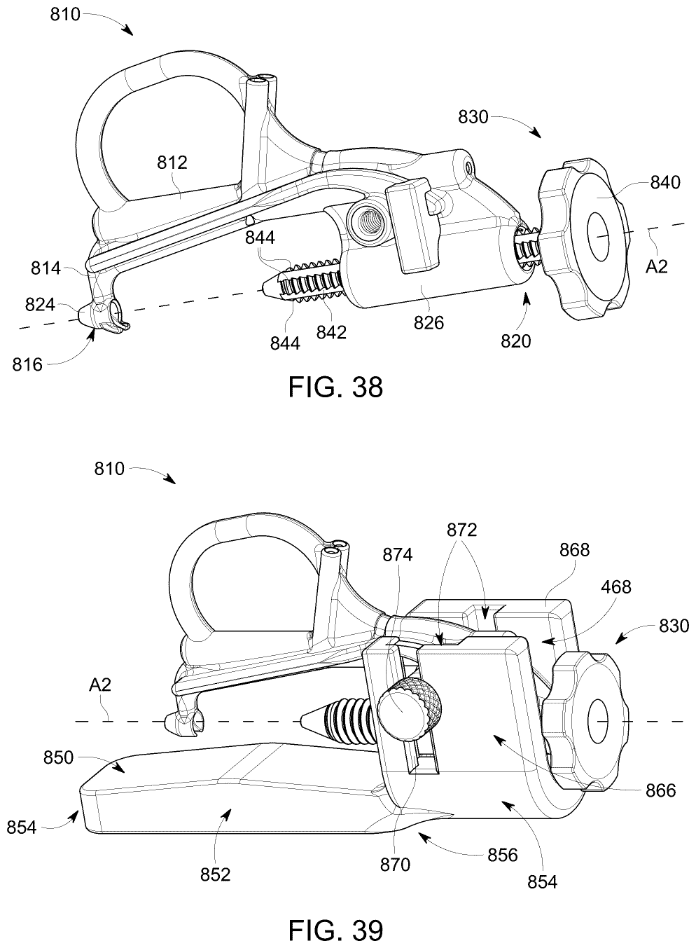

Another embodiment of a surgical instrument 810 is shown in FIGS. 33-38. The surgical instrument 810 has an elongated body 812 with a first leg 814 having a leading end 816, a second leg 818 having a trailing end 820 spaced from the leading end 816 along and aligned with an alignment axis A2, and a brace 822 that connects the first leg 814 to the second leg 818. The surgical instrument 810 includes a first positioning element 824 at the leading end 816, and a second positioning element 826 that is generally aligned with the first positioning element 824 and with the alignment axis A2. The first positioning element 824 may include a conical shaped inner surface 825 sized and shaped to receiving a head of a bone anchor (not shown). The surgical instrument may include a driving tool 830.

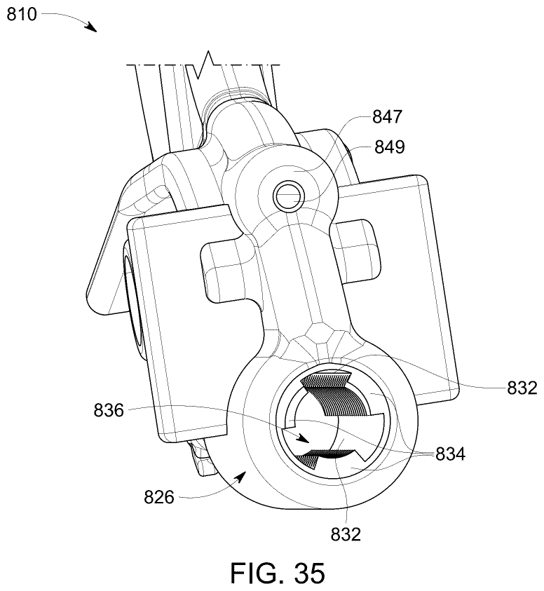

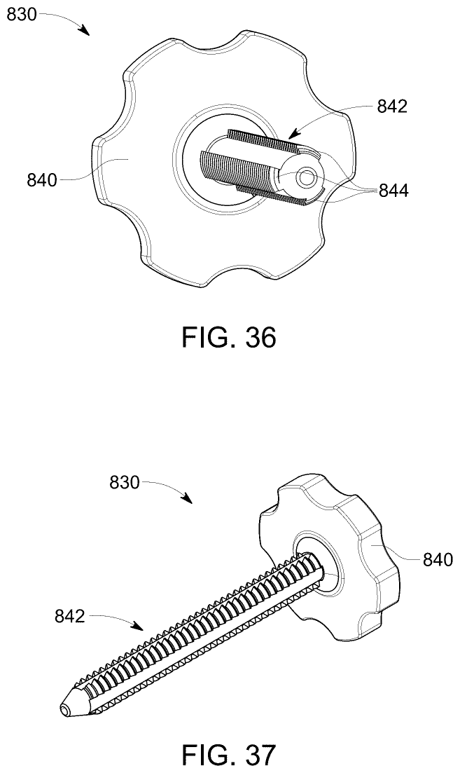

As shown in FIGS. 35-37, the second positioning element 826 includes a set of slots 832, a set of first threaded ridges 834 adjacent to the set of slots 832, respectively, and an inner channel 836 that extends along the alignment axis A2. The inner channel 836 is defined at least partially by an inner most surface 838 of the threaded ridges 834. The driving tool 830 as shown in FIGS. 36 and 37, includes head 840, a shaft 842, and a second set of thread ridges 844 that extends outwardly from the shaft 842. When the first set of threaded ridges 834 (of the first positioning element 824) are aligned with the set of slots 832 in the second positioning element 826, the shaft 842 of the driving tool 830 is axially translatable along the inner channel 836. Rotation of the driving tool 830 causes the second set of threaded ridges 844 to engage with the first set of threaded ridges 834 of the second positioning element 826, to threadably advance the driving tool 830 along the inner channel 836.

The surgical instrument 810 may further include a fixation guide member 846 carried by the brace 822. The fixation guide member 846 has one or more fixation guide channels 848 that extend in a direction that is angularly offset with respect to the alignment axis A2. The site line channel 849 is configured to receive a wire therethrough (wire not shown). The surgical instrument 810 includes a site line guide 847 with a site line guide channel 849 being substantially parallel with the alignment axis A2.

Referring to FIG. 39, an embodiment of instrument 810 includes a support tray 850. The support tray 850 is configured to support a plantar side of a toe. The support tray 850 has a support body 852 that has a first end 854 and a second end 856 opposite the first end 854 along an alignment axis A2, and an instrument engagement member 858 coupled to the second end 856 of the support body 852. The support body 852 has a lower surface 857, and an upper surface 860 spaced from the lower surface 857. The upper surface 860 has a first sloped portion 862 and a second sloped portion 864 that meets at a surface peak 867. Configured this way the first sloped portion 862 and the second sloped portion 864 are configured to support the toe.

The instrument engagement member 858 has a first wall 866, a second wall 868 spaced from the first wall 866, and a first slot 868 defined between the first and second walls 866 and 868. The first slot 4868 extends in a direction aligned with the alignment axis Al and is configured to receive an instrument therein. The instrument engagement member 858 has a second slot 870 that 1) extends through the first wall 866, and 2) an elongated indentation 872 in the second wall 868 and is offset with respect to the second slot 870. The instrument 810 further comprising a set screw 874 configured to extend through the second slot 870 to engage and fix the position of the instrument 810 in the instrument engagement member 858.

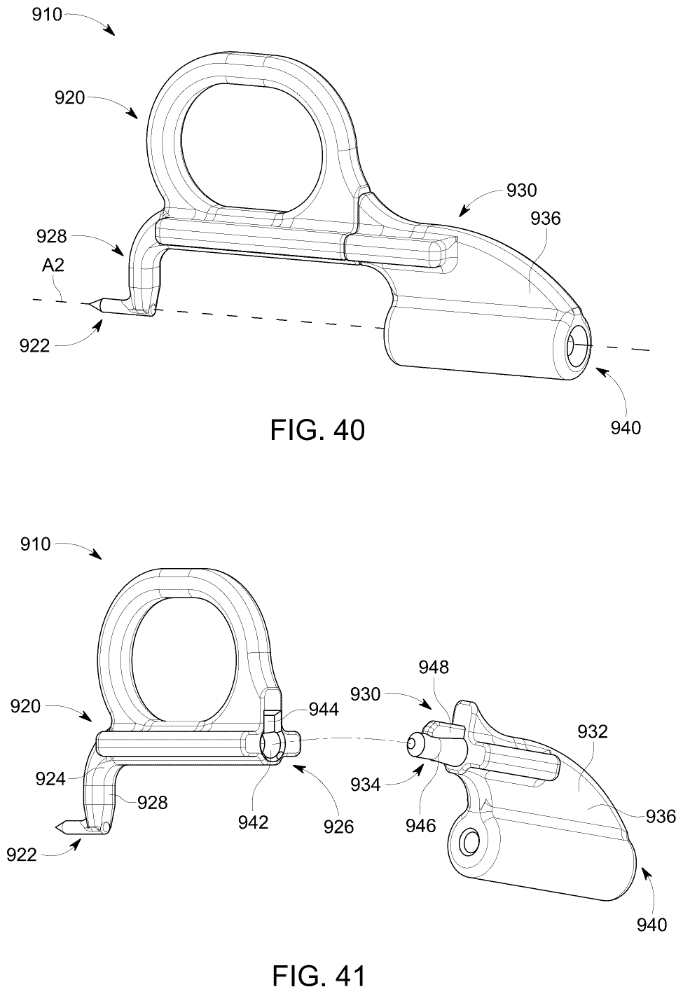

Referring to FIGS. 40 and 41, a bone fixation system according to another embodiment includes surgical instrument 910. The surgical instrument 910 includes a distal guide component 920 and a proximal guide component 930 that is configured to be removably coupled to the distal guide component 920. The distal guide component 920 has a distal component body 924 with a distal engagement element 926, and a distal leg 928 extending from the distal engagement element 926 and defining a leading end 922 of the distal guide component 920. The proximal guide component 930 has a proximal component body 932 with a proximal engagement element 934, and a proximal leg 936 extending from the proximal engagement element 938 and defining a trailing end 940 of the proximal guide component 930. The proximal engagement element 934 is configured to couple to the distal engagement element 926 to removably couple the distal guide component 920 and proximal guide component 922 together. The leading end 922 and trailing end 940 of the distal and proximal guide components 920, 930, respectively, are aligned along an alignment axis A2 when the distal and proximal components 920, 930 are coupled together.

In one example, the distal engagement element 926 is a bore 942 and a slot 944, and the proximal engagement element 934 is a rod 946 and a projection 948. Here, the rod 946 is configured to slidingly fit in the bore 942 and the projection 948 is configured to slide in the slot 944. Coupled this way, the proximal guide component 930 and the distal guide component 920 do not substantially rotate relative to each other when the proximal and distal components 930, 920 are coupled together. In an alternative example, the proximal engagement element 934 may be a bore and a slot, and the distal engagement element 926 is a rod and a projection. Accordingly, the distal engagement element is one of a bore or a rod, and the proximal engagement element is the other one of the bore of the rod. In yet another example, the distal engagement element includes a first magnet and the proximal engagement element includes a second magnet that is configured to temporarily lock the distal and proximal components together. Thus, the distal engagement element 926 and the proximal engagement element 934 may be any complementary features that, when coupled or engaged with each other, couple the proximal and distal components 930, 920 are together and also prevent rotation of the proximal and distal components 930, 920 relative to each other.

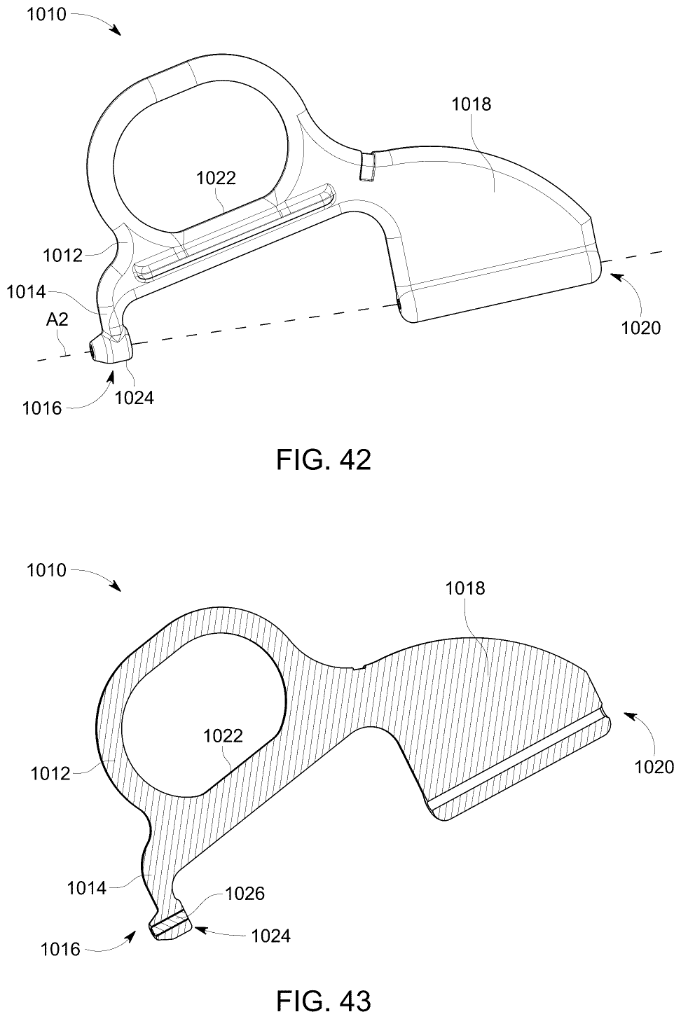

As shown in FIGS. 42 and 43, another embodiment is a bone fixation system, having a surgical instrument 1010 is illustrated. The surgical instrument 1010 has an elongated body 1012 with a first leg 1014 has a leading end 1016, a second leg 1018 has a trailing end 1020 spaced from the leading end 1016 along and aligned with an alignment axis A2, and a brace 1022 that connects the first leg 1014 to the second leg 1018. The surgical instrument 1010 has a first positioning element 1024 at the leading end 1016. The first positioning element 1024 has a first guide channel 1026. A second positioning element 1028 is coupled the second leg 1018 and is generally aligned with the first positioning element 1024. The second positioning element 1028 has a second guide channel 1030 aligned with the alignment axis A2 and the first guide channel 1026 of the first positioning element 1024. In addition, the surgical instrument 1010 includes a radiopaque element 1032 carried by the elongated body 1012. For example, radiopaque element 1032 may located at any portion of the elongated body 1012. The radiopaque element 1032 is configured to enable visualization of the surgical instrument 1010 during use. The radiopaque element 1032 may be polymer, insert, coating or additional device or material added to the elongated body 1012 to aid in visualization during the surgical procedure The surgical instrument further includes a gripping member 1033 carried by the brace.

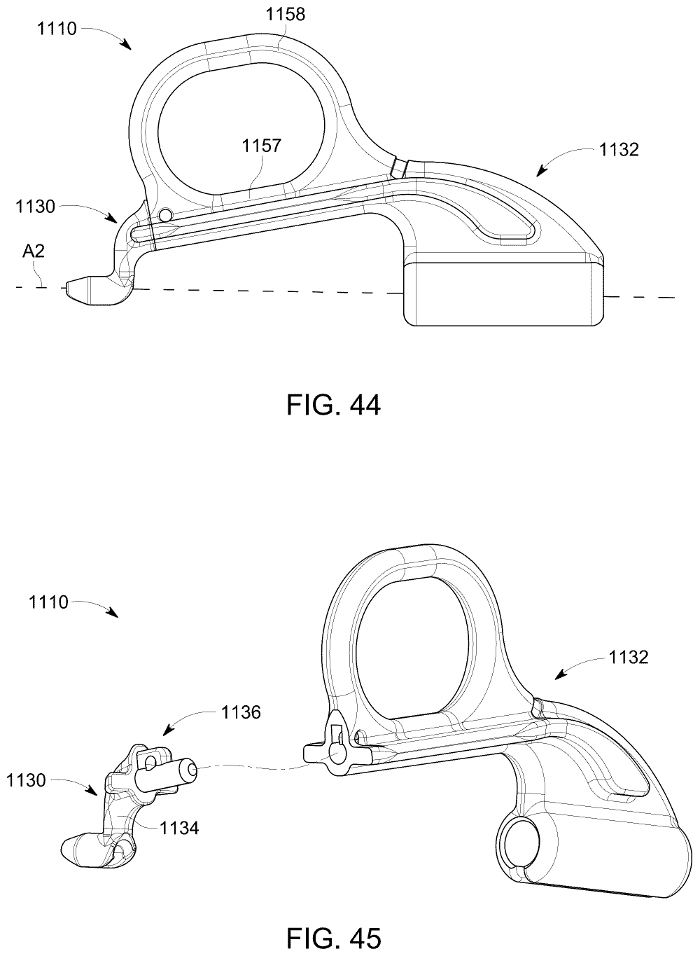

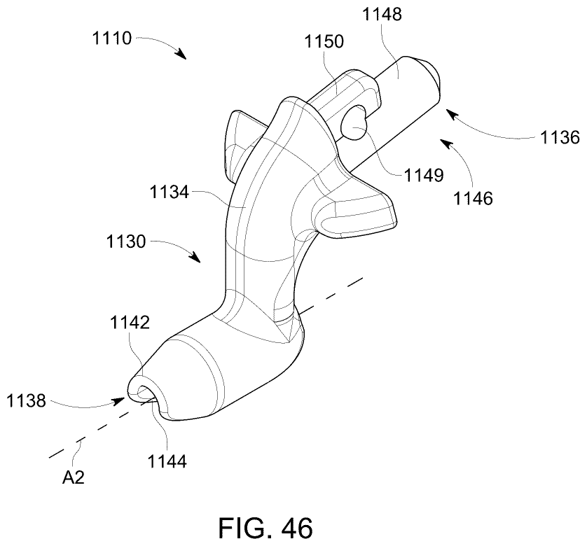

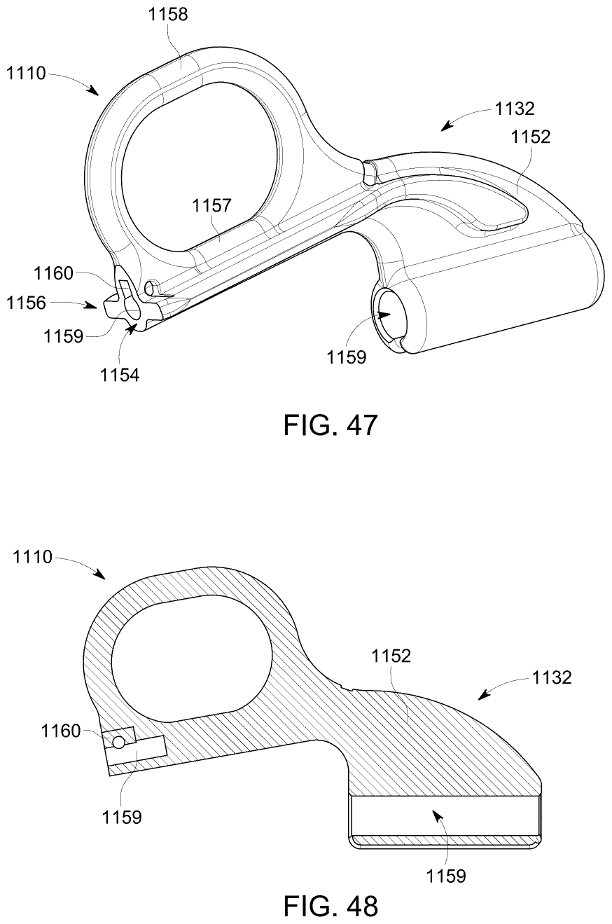

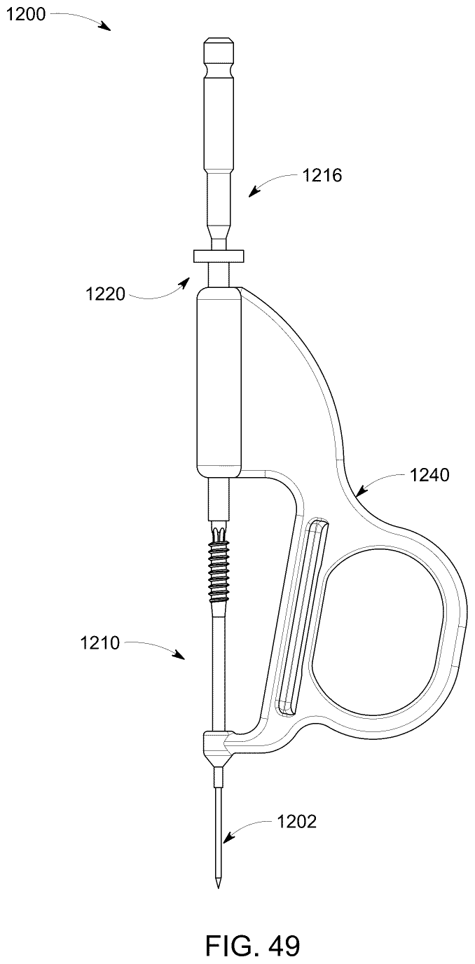

FIGS. 44-48 illustrate another embodiment of a surgical instrument 1110 for use in guiding an anchor or screws into position for bone fixation. The surgical instrument 1110 includes a distal guide component 1130 and a proximal guide component 1132 that is configured to be removably coupled to the distal guide component 1130 and are spaced apart from each other along an alignment axis A2.

Continuing with FIGS. 44-48, the distal guide component 1130 has a distal component body 1134 with a distal engagement element 1136 at its proximal end 1138, a guide channel 1140, with an open lower portion to form a U-shaped channel, and an apex 1142 and a distal-most end 1144 of the distal component body 1134. The rearward or proximal end 1146 of the distal engagement element 1136 includes engagement features for removable coupling to the proximal guide component 1132. In the example shown, distal engagement element 1136 may include a rod 1148, a projection 1150 extending from the rod 1148, and a through-hole 1149 that extends through the projection 1150 in a direction that is transverse, e.g. substantially perpendicular, to the alignment axis A2. The distal guide component 1130 may be formed from a radiopaque metal The distal guide component 1130 may be formed from a radiopaque metal to aid in visualization of the instrument during use.

The proximal guide component 1132 has a proximal component body 1152 with a proximal engagement element 1154 at its distal end 1156, a brace 1157 with a gripping member 1158 and a proximal guide channel formed through the component body 1152 and aligned along the alignment axis A2. The proximal guide component 1132 may be made from a radiopaque polymer.

Turning to FIG. 45, the proximal engagement element 1154 is configured to be removably coupled to the distal engagement element 1130 to enable removably coupling the distal and proximal components 1130, 1132 together. Here, for instance, the proximal engagement element 1154 is a bore 1159 and a slot 1160. Another through-hole 1162 extends through the proximal component body 1152 into the slot 1160. As shown in FIGS. 44 and 45, the instrument 1110 includes an engagement pin that extends through the through-holes 1149 on the distal guide component 1130 and the through-hole 1162 of the proximal guide component 1132 when the distal engagement element 1136 and the proximal engagement element 1154 are engaged or removably coupled together. The pin (not shown) keeps the two components fixed together until the user or surgeon removes that pin so that the proximal guide component 1132 can be removed from the distal guide component 1130. Coupled this way, the proximal guide component 1132 and the distal guide component 1130 are coupled together as shown in FIG. 44, the proximal guide component 1132 and the distal guide component 1130 do not substantially rotate relative to each other.

While the distal guide component is shown with a rod and a projection and the proximal guide component has a channel and a slot, the distal engagement element can be one of a bore or a rod, and the proximal engagement element may be the other one of the bore of the rod. While distal engagement element 1136 and the proximal engagement element 1154 are described has having a rod and a bore, respectively, the distal engagement element can be a bore and the proximal engagement element can be a rod. In yet another example, the distal engagement element may include a first magnet, and the proximal engagement element may include a second magnet that is configured to temporarily lock the distal and proximal components together. Furthermore, any other type of mating feature may be used to couple the two components together. During use, the distal and proximal guide components 1130, 1132 are typically kept together during the surgical procedure. The advantage here is that the surgeon can visualize the location of the distal engagement component on x-ray because it is radiopaque metal, and they get the benefit of having a clear view of the rest of the anatomy because the proximal component is a radiopaque polymer.

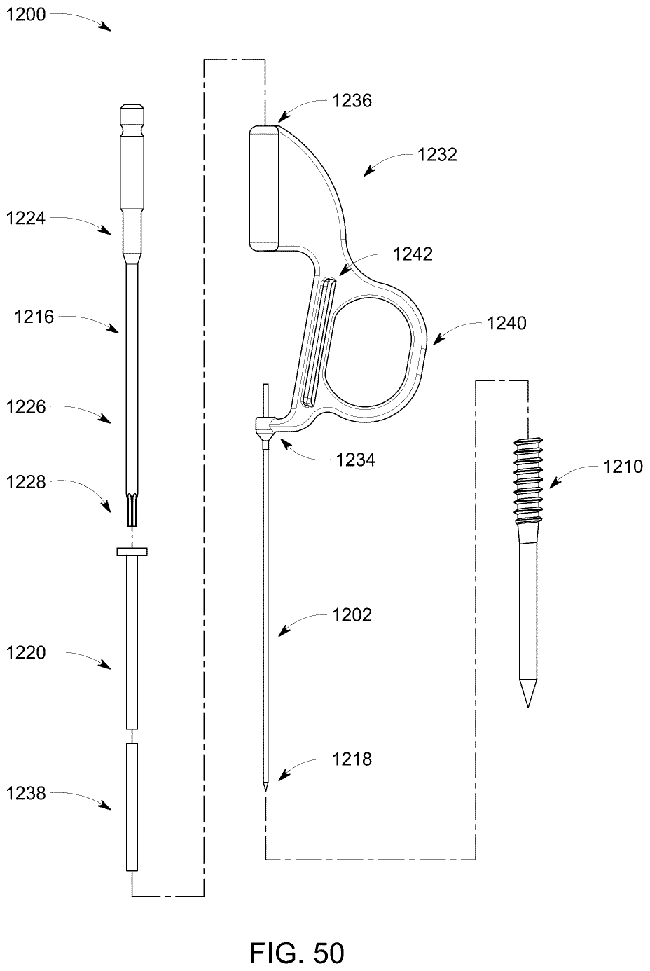

Referring now to FIGS. 49-51, there is shown a bone fixation system 1200. The bone fixation system 1200 includes a wire 1202, an implant anchor 1210, a moveable sleeve 1220, and surgical guide 1232. In the illustrated embodiment, the wire 1202 includes a sharp distal. The wire 1202 is insertable into the cannulation so that the sharp distal tip 1218 extends out of the anchor distal end 1216. The wire 1212 may be a k-wire.

The movable sleeve 1220 having an internal channel (not shown) and a driver 1222. The driver has a rear engagement portion 1224, a shaft 1226, and an engagement end 1228 on a distal end 1230 of the shaft 1226. The driver may include a cannulation (not shown). The engagement end 1228 is configured to engage the head 1212 of the implant anchor 1210. The driver 1222 may be inserted into the internal channel of the movable sleeve 1220 such that the movable sleeve 1220 is movable along the shaft 1226 of the driver 1222.

The surgical guide 1232 is configured for positioning the implant 1210 and wire 1202. The guide 1232 includes a forward end 1234 configured to slidingly mate with the shaft 1216 of the implant anchor 1210, a rearward end 1236 having an engagement body. The guide 1232 includes a guide member 1238 that is positioned within the engagement body. The guide member 1238 has a channel (not shown) configured to receive a portion of the driver 1222. As shown, the forward end 1234 can includes a gripping member 1240 to facilitate positioning of the guide. Specifically, the gripping member facilitates stability of the guide position during implantation of the wire. In the illustrated embodiment, the guide 1232 includes a curved elongated body 1242. The elongated body 1242 can be formed with a plurality of segmented portions having different cross-sectional dimensions to facilitate implantation of a wire 1202 and removal of the guide 1232 subsequently thereafter. The guide 1232 can be manufactured from a number of materials including nitinol, titanium alloys, non-titanium alloys, or other polymeric materials, e.g., plastics, plastic composites, polyetheretherketone (PEEK), and ceramics such as silicon nitride, zirconium oxide, silver oxide, and other suitable materials for facilitating detachment of the guide after implantation.

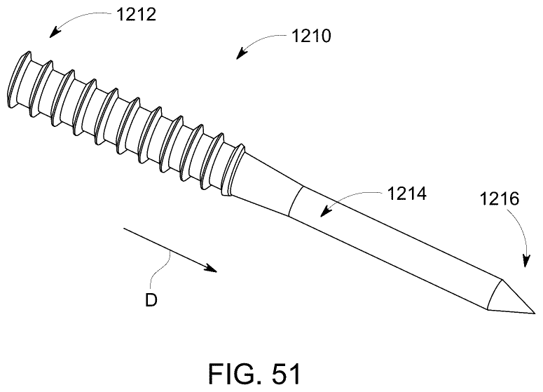

Referring now to FIG. 51, the implant anchor 1210 has a head 1212, a shaft 1214 that extends from the head 1212 in a distal direction D to define an anchor distal end 1216, and a cannulation (not numbered) that extends through the implant anchor 1210. The implant anchor 1210 has a proximal portion (not numbered) that is threaded while the entirety of the shaft 1214 that extends from the proximal portion is smooth and an unthreaded to its distal end 1216. The threaded portion of the implant anchor 1212 is configured to threadedly secure the implant anchor to the bone. Specifically, the threaded portions may be sized and shaped to be threadedly engaged within a bone canal or bore drilled into the bone prior to implantation of the implant anchor. The unthreaded portion of the shaft 1214 is flexible such that it can be bent out of alignment with the proximal end during use as needed. Furthermore, the shaft may include a blunt tip or a sharp tip at the distal end 1216.

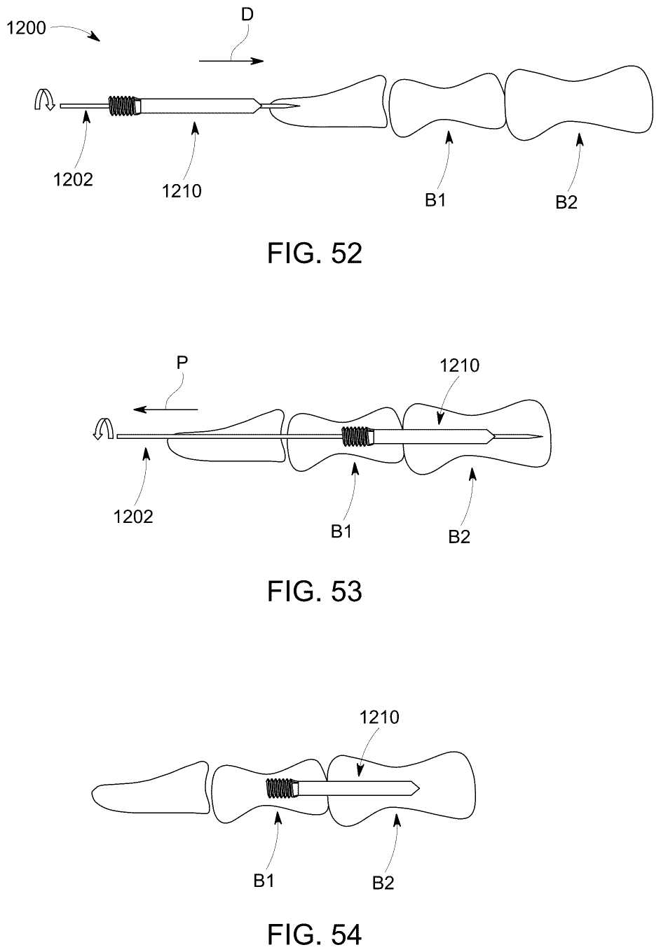

Referring to FIGS. 52-54, there is shown a bone fixation system 1200 used to fix to bone segments B1 and B2 together according to an embodiment of the present disclosure. The wire 1202 is insertable into the cannulation of the implant anchor 1210 so that the sharp distal tip 1218 extends out of the anchor distal end 1216. The implant anchor 1210 and wire 1202 assembly may be inserted through a bone into engagement with the first bone segment B1 and second bone segment B2 along a distal direction D. As shown in FIG. 52, the implant-wire assembly is inserted through an adjacent bone segment toward the desired fixation site. Torque may be applied the implant-wire assembly with a driver 1216 (not shown) to threadably engage the proximal threaded portion of the implant anchor 1210 with the bone. When the implant-wire assembly is place, the wire can be removed from the implant anchor 1210 in a proximal direction P. leaving the implant anchor 1210 engaged with the bone segments B1 and B2, as shown in FIGS. 53 and 54. Thus, the wire is configured to be coupled to the driver in an engaged state and uncoupled and retracted from the driver in a disengaged state when the implant anchor is implanted (as shown in FIG. 54).

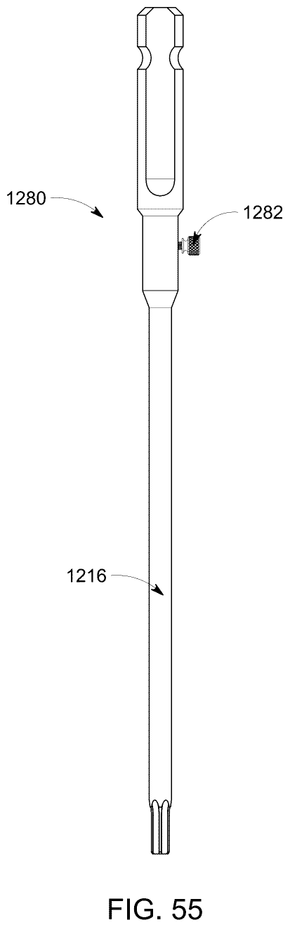

Referring to FIG. 55, there a driver 1280 used with bone fixation system 1200. The driver 1280 includes and a locking element 1282 coupled to the driver 1280. The driver 1280 includes the same features as the driver 1216 described above and those common features are not called out in FIG. 55 for easer of illustration. However, the driver 1280 further includes a bore (not numbered) positioned on the shaft 1226 and extending to the cannulation (not shown). The locking element 1282 may be threadably coupled to shaft inside the bore. The locking element 1282 itself includes a head and a threaded shaft that engages the inner surface of the bone and extends through to the cannulation. The locking element 1282 is configured to selectively lock a wire 1202 (not shown) inserted into the cannulation in place. The locking element 1282 is further configured to unlock the wire 1202 when the wire hits a desired anatomic depth, for example, as shown in FIG. 54.

Wherever possible, the same or like reference numbers are used throughout the drawings to refer to the same or like features. It should be noted that the drawings are in simplified schematic form and are not drawn to precise scale. Certain terminology used in the description is for convenience only and is not limiting. Directional terms such as top, bottom, left, right, above, below and diagonal, are used with respect to the accompanying drawings. The term “distal” shall mean away from the center of a body. The term “proximal” shall mean closer towards the center of a body and/or away from the “distal” end. The words “inwardly” and “outwardly” refer to directions toward and away from, respectively, the geometric center of the identified element and designated parts thereof. Such directional terms used in conjunction with the following description of the drawings should not be construed to limit the scope of the present disclosure in any manner not explicitly set forth. Additionally, the term “a,” as used in the specification, means “at least one.” The terminology includes the words above specifically mentioned, derivatives thereof, and words of similar import.

“Substantially” as used herein shall mean considerable in extent, largely but not wholly that which is specified, or an appropriate variation therefrom as is acceptable within the field of art. “Exemplary” as used herein shall mean serving as an example.

Furthermore, the described features, advantages and characteristics of exemplary embodiments may be combined in any suitable manner in one or more embodiments. One skilled in the art will recognize, in light of the description herein, that the exemplary embodiments can be practiced without one or more of the specific features or advantages of a particular embodiment. In other instances, additional features and advantages may be recognized in certain embodiments that may not be present in all embodiments of the present disclosure.

While the disclosure is described herein, using a limited number of embodiments, these specific embodiments are not intended to limit the scope of the disclosure as otherwise described and claimed herein. The precise arrangement of various elements and order of the steps of articles and methods described herein are not to be considered limiting. For instance, although the steps of the methods are described with reference to sequential series of reference signs and progression of the blocks in the figures, the method can be implemented in an order as desired.

1-34. (canceled)

35. A bone fixation system, comprising:
a fixation plate having an inferior surface, a superior surface opposite the inferior surface along a transverse direction, a middle portion configured to span a joint, a first fixation portion extending from and angularly offset with respect to the middle portion, and a second fixation portion extending from and angularly offset with respect to the middle portion,
the middle portion having at least one engagement member, the at least one engagement member configured to engage an aiming jig,
the first fixation portion having one or more through-holes that extend from the superior surface to the inferior surface,
the second fixation portion having one or more through-holes that extend from the superior surface to the inferior surface; and
one or more anchors configured to be inserted into the one or more through-holes to engage tissue.

36. The bone fixation system according to claim 35, wherein the one or more through-holes of the first fixation portion extends along a through-hole axis and include an upper portion that is tapered inward toward the through-hole axis.

37. The bone fixation system according to claim 35, wherein the one or more through-holes of the second fixation portion extends along a through-hole axis and include an upper portion that is tapered inward toward the through-hole axis.

38. The bone fixation system according to claim 35, wherein the one or more through-holes of the first fixation portion include two through-holes, wherein the one or more through-holes of the second fixation portion include two through-holes.

39. The bone fixation system according to claim 35, wherein the fixation plate has a first end at the first fixation portion that is tapered, and a second end at the second fixation portion that is tapered, wherein the tapered first and second ends are configured to guide the fixation plate between skin and bone proximate a joint.

40. A system, comprising:
an aiming jig having an inferior surface, a superior surface, a middle portion, a first guide portion extending from and angularly offset with respect to the middle portion, and a second guide portion extending from and angularly offset with respect to the middle portion,
the middle portion including an engagement member configured to facilitate temporary coupling to a fixation plate,
the first guide portion having one or more channels that extend from the superior surface to the inferior surface,
the second guide portion having one or more channels that extend from the superior surface to the inferior surface, wherein the one or more channels on the first guide portion and the second guide portion configured to receive therethrough a wire;
an arm extension coupled to the middle portion of the aiming jig, the arm extension having a guide member having a channel that extends therethrough, the channel configured to receive a wire therethrough; and
a threaded post configured to engage the engagement member and the fixation plate to couple the aiming jig to the fixation plate.

41. The system according to claim 40, wherein the one or more channels of the first guide portion include two channels, wherein the one or more channels of the second guide portion include two channels.

42-44. (canceled)

45. A system, comprising:
a surgical instrument including a preparation device body, the preparation device body having a proximal end, a distal end spaced from the proximal end, a tissue facing surface to place around tissue, an upper surface spaced from the tissue facing surface, and a slot extending into the preparation device body, wherein the slot extends from the upper surface to the tissue facing surface; and
an osteotomy block coupled to the preparation device body and an osteotomy slot that extends through the osteotomy block and aligned with the slot of the preparation device body.

46. The system according to claim 45, further comprising a block guide channel aligned with the osteotomy slot configured to receive therein a wire.

47. The system according to claim 45, wherein the surgical instrument comprises a reduction member coupled to the preparation device body, the reduction member including a reduction block, a threaded hole in the reduction block, a support block extending form the upper surface of the preparation device body, and a threaded actuator threadably coupled to the support block and the reduction block, wherein actuation of the actuator causes the reduction block to translate in a reduction slot in the preparation device body in a direction toward or away from the support block.

48. The system according to claim 45, further comprising a fixation guide channel along the tissue facing surface, the fixation guide channel configured to receive therethrough a fixation device.

49. The system according to claim 48, further comprising a site line guide channel along an upper surface of the preparation device body, the site line guide channel being substantially aligned with the fixation guide channel and configured to receive therethrough a radiopaque wire.

50-92. (canceled)

93. The bone fixation system according to claim 35, wherein:
a central axis of the one or more through-holes of the first fixation portion is non-parallel with a central axis of the one or more through-holes of the second fixation portion;
the central axis of the one or more through-holes of the first fixation portion is non-parallel with a central axis of an opening of the at least one engagement member.

94. The bone fixation system according to claim 93, wherein the opening of the at least one engagement member is a threaded opening, and the threaded opening is configured to receive a threaded post to secure the fixation plate to the aiming jig.

95. The bone fixation system according to claim 35, wherein the at least one engagement member is configured to engage with an engagement member of the aiming jig.

96. The bone fixation system according to claim 95, wherein the at least one engagement member is configured to receive an engage with a protrusion of the engagement member of the aiming jig.

97. The bone fixation system according to claim 35, wherein the at least one engagement member comprises an engagement surface that is offset relative to the superior surface.

98. The bone fixation system according to claim 97, wherein the engagement surface is offset relative to the superior surface in a direction towards the inferior surface.

99. The bone fixation system according to claim 97, wherein:
the aiming jig includes an inferior surface, a superior surface, a middle portion, a first guide portion extending from and angularly offset with respect to the middle portion, and a second guide portion extending from and angularly offset with respect to the middle portion;
the middle portion of the aiming jig includes the engagement member configured to engage with the at least one engagement member of the fixation plate;
the first guide portion of the aiming jig has one or more channels that extend from the superior surface to the inferior surface of the aiming jig;
the second guide portion of the aiming jig has one or more channels that extend from the superior surface to the inferior surface of the aiming jig, the one or more channels on the first guide portion and the second guide portion being configured to receive therethrough a wire;
the middle portion of the aiming jig is coupled to an arm extension, the arm extension having a guide member having a channel that extends therethrough, the channel configured to receive a wire therethrough; and
a threaded post is configured to engage the engagement member of the aiming jig and the fixation plate to couple the aiming jig to the fixation plate.

100. The system according to claim 40, wherein the arm extension includes an L-shaped arm portion extending between the guide member and the middle portion of the aiming jig.
